(19) United States
(12) Patent Application Publication
(10) Pub. No.: US 2009/0155896 A1
(43) Pub. Date: Jun. 18, 2009

(54) Human Cancer Suppressor Gene, Protein Encoded Therein, Expression Vector Containing The Same, And Cell Transformed By The Vector

(76) Inventors: Hyun-Kee Kim, Seoul (KR); Jin-Woo Kim, Seoul (KR)

Correspondence Address: HAMRE, SCHUMANN, MUELLER & LARSON, P.C., P.O. BOX 2902, MINNEAPOLIS, MN, 55402-0902, US

(21) Appl. No.: 11/793,866

(22) PCT Filed: Dec. 22, 2005
(86) PCT No.: PCT/KR2005/004477

§ 371 Date: Nov. 13, 2007

(30) Foreign Application Priority Data
Dec. 22, 2004 (KR) 10-2004-110326

(51) Int. Cl. C12N 5/00; C07K 16/00; C07H 21/04; C12N 15/00

(52) U.S. Cl. 435/325; 530/350; 536/23.5; 435/320.1; 435/252.3; 435/252.33

(57) ABSTRACT

Disclosed are a human cancer suppressor gene, a protein encoded therein, an expression vector containing the same, and a cell transformed by the vector. The gene of the present invention can be used for diagnosing, preventing and treating the human cancers.

### TECHNICAL FIELD

The present invention relates to a human cancer suppressor gene, a protein encoded therein, an expression vector containing the same, and a cell transformed by the vector.

### BACKGROUND ART

Tumor suppressor gene products function to suppress normal cells from being transformed into certain cancer cells, and therefore loss of this function of the tumor suppressor gene products allows the normal cells to become malignant transformants (Klein, G., FASEB J., 7, 821-825 (1993)). In order to allow cancer cells to grow into a cancer, the cells should lose a function to control the normal copy number of a tumor suppressor gene. It was found that modification in a coding sequence of a p53 tumor suppressor gene is one of the most general genetic changes in the human cancers (Bishop, J. M., Cell, 64, 235-248 (1991); and Weinberg, R. A., Science, 254, 1138-1146 (1991)).

However, it was estimated that only some of breast cancer tissues exhibited a p53 mutation because the reported p53 mutation was in a range of 30% in the breast cancer (Keen, J. C. & Davidson, N. E., Cancer, 97, 825-833 (2003)) and Borresen-Dale, A-L., Human Mutation, 21, 292-300 (2003)).

The p53 mutation accounts for at least 50% of liver cancer especially in the region exposed to aflatoxin B1 or having a high frequency of infection by hepatitis B virus, and it is mainly characterized by a missense mutation at a codon 249 in the p53 tumor gene (Montesano, R. et al., J. Natl. Cancer Inst., 89, 1844-1851 (1997); Szymanska, K. & Hainaut, P. Acta Biochimica Polonica, 50, 231-238 (2003)). However, the p53 mutation was nothing but a range of 30% of breast cancer in U.S. and Western Europe, and there is no hot spot in which such mutation occurs more frequently (Szymanska, K. & Hainaut, P. Acta Biochimica Polonica, 50, 231-238 (2003)).

Accordingly, the present inventors have ardently attempted to separate a novel tumor suppressor gene from normal breast tissues using an mRNA differential display (DD) method for effectively displaying genes differentially expressed between a normal breast tissue and a breast cancer, or between a normal liver tissue and a liver cancer (Liang, P. and Pardee, A. B., Science, 257, 967-971 (1992); and Liang, P. et al., Cancer Res., 52, 6966-6968 (1993)).

### DISCLOSURE OF INVENTION

### Technical Problem

Accordingly, the present invention is designed to solve the problems of the prior art, and therefore it is an object of the present invention to provide a novel human cancer suppressor gene.

It is another object of the present invention to provide a cancer suppressor protein coded by the gene.

It is still another object of the present invention to provide an expression vector containing the gene.

It is yet another object of the present invention to provide a cell transformed by the expression vector.

### Technical Solution

In order to accomplish the above object, the present invention provides a human cancer suppressor gene (growth-inhibiting gene 12; also referred to as GIG12) having a DNA sequence set forth in SEQ ID NO: 1.

In order to accomplish the other object, the present invention provides a human cancer suppressor protein having an amino acid sequence set forth in SEQ ID NO: 2, which is encoded by the GIG12 gene.

The present invention also provides a human cancer suppressor gene (growth-inhibiting gene 17; also referred to as GIG17) having a DNA sequence set forth in SEQ ID NO: 5.

The present invention provides a human cancer suppressor protein having an amino acid sequence set forth in SEQ ID NO: 6, which is encoded by the GIG17 gene.

The present invention also provides a human cancer suppressor gene (growth-inhibiting gene 19; also referred to as GIG19) having a DNA sequence set forth in SEQ ID NO: 9.

The present invention provides a human cancer suppressor protein having an amino acid sequence set forth in SEQ ID NO: 10, which is encoded by the GIG19 gene.

The present invention also provides a human cancer suppressor gene (growth-inhibiting gene 20; also referred to as GIG20) having a DNA sequence set forth in SEQ ID NO: 13.

The present invention provides a human cancer suppressor protein having an amino acid sequence set forth in SEQ ID NO: 14, which is encoded by the GIG20 gene.

The present invention also provides a human cancer suppressor gene (growth-inhibiting gene 22; also referred to as GIG22) having a DNA sequence set forth in SEQ ID NO: 17.

The present invention provides a human cancer suppressor protein having an amino acid sequence set forth in SEQ ID NO: 18, which is encoded by the GIG22 gene.

The present invention also provides a human cancer suppressor gene (growth-inhibiting gene 25; also referred to as GIG25) having a DNA sequence set forth in SEQ ID NO: 21.

The present invention provides a human cancer suppressor protein having an amino acid sequence set forth in SEQ ID NO: 22, which is encoded by the GIG25 gene.

The present invention also provides a human cancer suppressor gene (growth-inhibiting gene 36; also referred to as GIG36) having a DNA sequence set forth in SEQ ID NO: 25.

The present invention provides a human cancer suppressor protein having an amino acid sequence set forth in SEQ ID NO: 26, which is encoded by the GIG36 gene.

The present invention also provides a human cancer suppressor gene (growth-inhibiting gene 2; also referred to as GIG2) having a DNA sequence set forth in SEQ ID NO: 29.

The present invention provides a human cancer suppressor protein having an amino acid sequence set forth in SEQ ID NO: 30, which is encoded by the GIG2 gene.

According to still another object, the present invention provides an expression vector containing each of the genes.

According to yet another object, the present invention provides a cell transformed by each of the expression vectors.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a gel diagram showing a PCR result using a 5′-13-mer random primer H-AP32 of SEQ ID NO: 3 and an anchored oligo-dT primer of SEQ ID NO: 4;

FIG. 2 is a gel diagram showing a PCR result using a 5′-13mer random primer H-AP7 of SEQ ID NO: 7 and an anchored oligo-dT primer of SEQ ID NO: 8;

FIG. 3 is a gel diagram showing a PCR result using a 5′-13mer random primer H-AP45 of SEQ ID NO: 11 and an anchored oligo-dT primer of SEQ ID NO: 12;

FIG. 4 is a gel diagram showing a PCR result using a 5′-13mer random primer H-AP40 of SEQ ID NO: 15 and an anchored oligo-dT primer of SEQ ID NO: 16;

FIG. 5 is a gel diagram showing a PCR result using a 5′-13mer random primer H-AP30 of SEQ ID NO: 19 and an anchored oligo-dT primer of SEQ ID NO: 20;

FIG. 6 is a gel diagram showing a PCR result using a 5′-13mer random primer H-AP40 of SEQ ID NO: 23 and an anchored oligo-dT primer of SEQ ID NO: 24;

FIG. 7 is a gel diagram showing a PCR result using a 5′-13mer random primer H-AP29 of SEQ ID NO: 27 and an anchored oligo-dT primer of SEQ ID NO: 28;

FIG. 8 is a gel diagram showing a PCR result using a 5′-13mer random primer H-AP32 of SEQ ID NO: 31 and an anchored oligo-dT primer of SEQ ID NO: 32;

FIG. 9 is a diagram showing a result that a gene product of the GIG12 is analyzed on SDS-PAGE;

FIG. 10 is a diagram showing a result that a gene product of the GIG17 is analyzed on SDS-PAGE;

FIG. 11 is a diagram showing a result that a gene product of the GIG19 is analyzed on SDS-PAGE;

FIG. 12 is a diagram showing a result that a gene product of the GIG20 is analyzed on SDS-PAGE;

FIG. 13 is a diagram showing a result that a gene product of the GIG22 is analyzed on SDS-PAGE;

FIG. 14 is a diagram showing a result that a gene product of the GIG25 is analyzed on SDS-PAGE;

FIG. 15 is a diagram showing a result that a gene product of the GIG-36 is analyzed on SDS-PAGE;

FIG. 16 is a diagram showing a result that a gene product of the GIG2 is analyzed on SDS-PAGE;

FIG. 17(a) is a diagram showing a northern blotting result that the GIG12 gene is differentially expressed in a normal breast tissue, a primary breast cancer tissue and a breast cancer cell line, and FIG. 17(b) is a diagram showing a northern blotting result obtained by hybridizing the same blot with β-actin probe;

FIG. 18(a) is a diagram showing a northern blotting result that the GIG17 gene is differentially expressed in a normal breast tissue, a primary breast cancer tissue and a breast cancer cell line, and FIG. 18(b) is a diagram showing a northern blotting result obtained by hybridizing the same blot with β-actin probe;

FIG. 19(a) is a diagram showing a northern blotting result that the GIG19 gene is differentially expressed in a normal breast tissue, a primary breast cancer tissue and a breast cancer cell line, and FIG. 19(b) is a diagram showing a northern blotting result obtained by hybridizing the same blot with β-actin probe;

FIG. 20(a) is a diagram showing a northern blotting result that the GIG20 gene is differentially expressed in a normal breast tissue, a primary breast cancer tissue and a breast cancer cell line, and FIG. 20(b) is a diagram showing a northern blotting result obtained by hybridizing the same blot with β-actin probe;

FIG. 21(a) is a diagram showing a northern blotting result that the GIG22 gene is differentially expressed in a normal breast tissue, a primary breast cancer tissue and a breast cancer cell line, and FIG. 21(b) is a diagram showing a northern blotting result obtained by hybridizing the same blot with β-actin probe;

FIG. 22(a) is a diagram showing a northern blotting result that the GIG25 gene is differentially expressed in a normal breast tissue, a primary breast cancer tissue and a breast cancer cell line, and FIG. 22(b) is a diagram showing a northern blotting result obtained by hybridizing the same blot with β-actin probe;

FIG. 23(a) is a diagram showing a northern blotting result that the GIG36 gene is differentially expressed in a normal breast tissue, a primary breast cancer tissue and a breast cancer cell line, and FIG. 23(b) is a diagram showing a northern blotting result obtained by hybridizing the same blot with β-actin probe;

FIG. 24(a) is a diagram showing a northern blotting result that the GIG2 gene is differentially expressed in a normal lung tissue, a primary lung cancer tissue, a metastatic lung cancer tissue and a lung cancer cell line, and FIG. 24(b) is a diagram showing a northern blotting result obtained by hybridizing the same blot with β-actin probe;

FIG. 25(a) is a diagram showing a northern blotting result that the GIG12 gene is differentially expressed in various normal tissues, and FIG. 25(b) is a diagram showing a northern blotting result obtained by hybridizing the same blot with β-actin probe;

FIG. 26(a) is a diagram showing a northern blotting result that the GIG17 gene is differentially expressed in various normal tissues, and FIG. 26(b) is a diagram showing a northern blotting result obtained by hybridizing the same blot with β-actin probe;

FIG. 27(a) is a diagram showing a northern blotting result that the GIG19 gene is differentially expressed in various normal tissues, and FIG. 27(b) is a diagram showing a northern blotting result obtained by hybridizing the same blot with β-actin probe;

FIG. 28(a) is a diagram showing a northern blotting result that the GIG20 gene is differentially expressed in various normal tissues, and FIG. 28(b) is a diagram showing a northern blotting result obtained by hybridizing the same blot with β-actin probe;

FIG. 29(a) is a diagram showing a northern blotting result that the GIG22 gene is differentially expressed in various normal tissues, and FIG. 29(b) is a diagram showing a northern blotting result obtained by hybridizing the same blot with β-actin probe;

FIG. 30(a) is a diagram showing a northern blotting result that the GIG25 gene is differentially expressed in various normal tissues, and FIG. 30(b) is a diagram showing a northern blotting result obtained by hybridizing the same blot with β-actin probe;

FIG. 31(a) is a diagram showing a northern blotting result that the GIG36 gene is differentially expressed in various normal tissues, and FIG. 31(b) is a diagram showing a northern blotting result obtained by hybridizing the same blot with β-actin probe;

FIG. 32(a) is a diagram showing a northern blotting result that the GIG2 gene is differentially expressed in various normal tissues, and FIG. 32(b) is a diagram showing a northern blotting result obtained by hybridizing the same blot with β-actin probe;

FIG. 33(a) is a diagram showing a northern blotting result that the GIG12 gene is differentially expressed in various cancer cell lines, and FIG. 33(b) is a diagram showing a northern blotting result obtained by hybridizing the same blot with β-actin probe;

FIG. 34(a) is a diagram showing a northern blotting result that the GIG17 gene is differentially expressed in various cancer cell lines, and FIG. 34(b) is a diagram showing a northern blotting result obtained by hybridizing the same blot with β-actin probe;

FIG. 35(a) is a diagram showing a northern blotting result that the GIG19 gene is differentially expressed in various cancer cell lines, and FIG. 35(b) is a diagram showing a northern blotting result obtained by hybridizing the same blot with β-actin probe;

FIG. 36(a) is a diagram showing a northern blotting result that the GIG20 gene is differentially expressed in various cancer cell lines, and FIG. 36(b) is a diagram showing a northern blotting result obtained by hybridizing the same blot with β-actin probe;

FIG. 37(a) is a diagram showing a northern blotting result that the GIG22 gene is differentially expressed in various cancer cell lines, and FIG. 37(b) is a diagram showing a northern blotting result obtained by hybridizing the same blot with β-actin probe;

FIG. 38(a) is a diagram showing a northern blotting result that the GIG25 gene is differentially expressed in various cancer cell lines, and FIG. 38(b) is a diagram showing a northern blotting result obtained by hybridizing the same blot with β-actin probe;

FIG. 39(a) is a diagram showing a northern blotting result that the GIG36 gene is differentially expressed in various cancer cell lines, and FIG. 39(b) is a diagram showing a northern blotting result obtained by hybridizing the same blot with β-actin probe;

FIG. 40(a) is a diagram showing a northern blotting result that the GIG2 gene is differentially expressed in various cancer cell lines, and FIG. 40(b) is a diagram showing a northern blotting result obtained by hybridizing the same blot with β-actin probe;

FIG. 41 is a graph showing growth curves of the wild-type MCF-7 cell, the MCF-7 breast cancer cell transfected by the GIG12 gene, and the MCF-7 cell transfected by the expression vector pcDNA3.1;

FIG. 42 is a graph showing growth curves of the wild-type HepG2 liver cancer cell line, the HepG2 liver cancer cell transfected by the GIG17 gene, and the HepG2 cell transfected by the expression vector pcDNA3.1;

FIG. 43 is a graph showing growth curves of the wild-type HepG2 liver cancer cell line, the HepG2 liver cancer cell transfected by the GIG19 gene, and the HepG2 cell transfected by the expression vector pcDNA3.1;

FIG. 44 is a graph showing growth curves of the wild-type HepG2 liver cancer cell line, the HepG2 liver cancer cell transfected by the GIG20 gene, and the HepG2 cell transfected by the expression vector pcDNA3.1;

FIG. 45 is a graph showing growth curves of the wild-type HepG2 liver cancer cell line, the HepG2 liver cancer cell transfected by the GIG22 gene, and the HepG2 cell transfected by the expression vector pcDNA3.1;

FIG. 46 is a graph showing growth curves of the wild-type HepG2 liver cancer cell line, the HepG2 liver cancer cell transfected by the GIG25 gene, and the HepG2 cell transfected by the expression vector pcDNA3.1;

FIG. 47 is a graph showing growth curves of the wild-type HepG2 liver cancer cell line, the HepG2 liver cancer cell transfected by the GIG36 gene, and the HepG2 cell transfected by the expression vector pcDNA3.1; and

FIG. 48 is a graph showing growth curves h of the wild-type A549 lung cancer cell line, the A549 lung cancer cell transfected by the GIG2 gene, and the A549 cell transfected by the expression vector pcDNA3.1.

### BEST MODE

Hereinafter, preferred embodiments of the present invention will be described in detail referring to the accompanying drawings.

1. GIG12

The gene of the present invention is a human cancer suppressor gene 36 (GIG36) showing a DNA sequence of SEQ ID NO: 1, which has been deposited with Accession No. AY493417 into the GenBank database of U.S. National Institutes of Health (NIH) (Publication Date: Mar. 1, 2005), and some DNA sequence of the deposited gene is identical with that of the lactotransferrin deposited with Accession No. NM_{—}002343 into the database. The lactotransferrin is abundantly distributed mainly in milk and serum, and its function has been known as only a carrier of ferric ions (Kanyshkova, G. T., et al., Biochemistry (Moscow), 66, 1-7 (2001)). At the same time, it was found that the lactotransferrin has only a strong antibacterial activity (Oppenheimer, J. S. J. Nutr., 131, 6165-6335 (2001); Shugars, C. D., et al., Gerontology, 47, 246-253 (2001)).

Contrary to the functions as reported previously, it was however found from this study result that the lactotransferrin is closely associated with various carcinogenesis. It was also found that the GIG12 tumor suppressor gene was not at all expressed in various human tumors including the breast cancer, while its expression was increased in various normal tissues.

The DNA sequence of SEQ ID NO: 1 has one open reading frame (ORF) corresponding to base positions from 111 to 2246 of the DNA sequence (Base positions from 2244 to 2246 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 711 amino acid residues, and has an amino acid sequence of SEQ ID NO: 2 and a molecular weight of approximately 78 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and the protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 1. As another example, a 680-bp cDNA fragment, which is not expressed in the cancer tissue or the cancer cell line but differentially expressed only in the normal tissue, may be obtained by carrying out a reverse transcription polymerase chain reaction (RT-PCR) on the total RNAs extracted from a normal tissue, and a cancer tissue or a cancer cell line using a random primer H-AP32 of SEQ ID NO: 3 (5′-AAGCTTCCTGCAA-3′) and an anchored oligo-dT primer of SEQ ID NO: 4 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a MCF-7 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG12 cDNA into an expression vector pcDNA3.1 (Invitrogen, U.S.), and then transformed Escherichia coli DH5α with the resultant expression vector to obtain a transformant, which was then named E. coli DH5α/GIG12/pcDNA3.1, and deposited with Accession No. KCTC 10642BP into Korean Collection for Type Cultures on May 24, 2004.

It is regarded that the gene of the present invention is overexpressed in normal tissues, preferably breast, lungs, thymus, liver, skeletal muscles, kidney, spleen, heart, placenta, and peripheral bloods, to suppress carcinogenesis. The gene of the present invention is mainly overexpressed in these tissues as an mRNA transcript having a size of approximately 2.4 kb. Especially, the gene of the present invention is differentially expressed only in the normal tissues. For example, the gene of the present invention is not expressed in the cancer tissues and the cancer cells such as the breast cancer tissue, the breast cancer cell line MCF-7, etc., but differentially expressed only in the normal tissues.

The cancer cell line into which the genes of the present invention were introduced showed a high mortality, and therefore the gene of the present invention may be effectively used for treatment and prevention of the cancer.

2. GIG17

The gene of the present invention is a human cancer suppressor gene 17 (GIG17) having a DNA sequence of SEQ ID NO: 5, which has been deposited with Accession No. AY544122 into the GenBank database of U.S. National Institutes of Health (NIH) (Publication Date: Dec. 31, 2005), and the deposited gene has polymorphism that its 3 base pairs are different to a DNA sequence of the human fructose 1,6-bisphosphatase deposited with Accession No. M19922 into the database.

One of the most important reactions in a glucose metabolism is to hydrolyze fructose 1,6-bisphosphate into fructose-6-phosphate (Marcus, F. et al., Arch. Biol. Med. Exp., 20, 371-378 (1987); Okar, D, A. & Lange, A. J. Biofactors, 10, 1-14 (1999)). An enzyme that catalyzes the metabolism is the human fructose 1,6-bisphosphatase, which is present in all living organisms.

Contrary to the glucose metabolism as reported previously, it was however found from this study result that the GIG17 tumor suppressor gene was not at all expressed in various human tumors including the liver cancer, while its expression was significantly increased in various normal tissues.

The DNA sequence of SEQ ID NO: 5 has one open reading frame (ORF) corresponding to base positions from 88 to 1104 of the DNA sequence (Base positions from 1102 to 1104 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 338 amino acid residues, and has an amino acid sequence of SEQ ID NO: 6 and a molecular weight of approximately 37 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 5. As another example, a 250-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP7 of SEQ ID NO: 7 (5′-AAGCTTAACGAGG-3′) and an anchored oligo-dT primer of SEQ ID NO: 8 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a HepG2 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG17 cDNA into an expression vector pcDNA3.1 (Invitrogen, U.S.), and then transformed Escherichia coli DH5α with the resultant expression vector to obtain a transformant, which was then named E. coli DH5α/GIG17/pcDNA3.1, and deposited with Accession No. KCTC 10655BP into Korean Collection for Type Cultures on Jun. 14, 2004.

It is regarded that the gene of the present invention is overexpressed in normal tissues, preferably liver, kidney, spleen and lungs, to suppress carcinogenesis. It is also regarded that the gene of the present invention is suppressed even in leukemia, uterine cancer, malignant lymphoma, colon cancer and skin cancer to induce carcinogenesis. The gene of the present invention is mainly overexpressed in these tissues as an mRNA transcript having a size of approximately 1.3 kb. Especially, the gene of the present invention is differentially expressed only in the normal tissues. For example, the gene of the present invention is not expressed in the cancer tissues and the cancer cells such as the liver cancer tissue, the liver cancer cell line HepG2, etc., but differentially expressed only in the normal liver tissue.

The cancer cell line into which the genes of the present invention were introduced showed a high mortality, and therefore the gene of the present invention may be effectively used for treatment and prevention of the cancer.

3. GIG19

The gene of the present invention is a human cancer suppressor gene 19 (GIG19) having a DNA sequence of SEQ ID NO: 9, which has been deposited with Accession No. AY544123 into the GenBank database of U.S. National Institutes of Health (NIH) (Publication Date: Dec. 31, 2005), and the DNA sequence of the deposited gene is identical with those of the Homo sapiens alpha-1-microglobulin/bikunin precursor and the human mRNA for protein HC (alpha-1-microglobulin), deposited with Accession No. BC041593 and X04225 into the existing database, respectively. The alpha-1-microglobulin, also referred to as an HC protein, is a lipoprotein having an immunosuppressive effect (Akerstrom, B. et al., Biochimica Biophysica Acta, 1482, 172-184 (2002); Xu, S. & Venge, P., Biochimica Biophysica Acta, 1482, 298-307 (2002)). Contrary to the functions of the tumor suppressor gene as reported previously, it was however found from this study result that the GIG19 tumor suppressor gene was not at all expressed in the liver cancer, while its expression was significantly increased in various normal liver tissues.

The DNA sequence of SEQ ID NO: 9 has one open reading frame (ORF) corresponding to base positions from 61 to 1119 of the DNA sequence (Base positions from 59 to 61 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%; preferably at least 90%; and the most preferably at least 95%.

The protein expressed from the gene of the present invention consists of 352 amino acid residues, and has an amino acid sequence of SEQ ID NO: 10 and a molecular weight of approximately 39 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 9. As another example, a 281-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP40 of SEQ ID NO: 11 (5′-AAGCTTGTCAGCC-3′) and an anchored oligo-dT primer of SEQ ID NO: 12 (5′-AAGCTTTTTTTTTTTC-3′); and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a HepG2 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG19 cDNA into an expression vector pcDNA3.1 (Invitrogen, U.S.), and then transformed Escherichia coli DH5α with the resultant expression vector to obtain a transformant, which was then named E. coli DH5α/GIG19/pcDNA3.1, and deposited with Accession No. KCTC 10656BP into Korean Collection for Type Cultures on Jun. 14, 2004.

It is regarded that the gene of the present invention is overexpressed in normal tissues, preferably liver tissues, to suppress carcinogenesis. The gene of the present invention is mainly overexpressed in these tissues as an mRNA transcript having a size of approximately 1.2 kb. Especially, the gene of the present invention is differentially expressed in the normal tissues. For example, the gene of the present invention is not expressed in the cancer tissues and the cancer cells such as the liver cancer tissue, the liver cancer cell line HepG2, etc., but differentially expressed only in the normal liver tissue.

The liver cancer cell line into which the genes of the present invention were introduced showed a high mortality, and therefore the gene of the present invention may be effectively used for treatment and prevention of the cancer.

4. GIG20

The gene of the present invention is a human cancer suppressor gene 20 (GIG20) having a DNA sequence of SEQ ID NO: 13, which has been deposited with Accession No. AY544124 into the GenBank database of U.S. National Institutes of Health (NIH) (Publication Date: Dec. 31, 2005), and the DNA sequence of the deposited gene is identical with that of the Homo sapiens albumin deposited with Accession No. BC041789 into the existing database. It has been known that the albumin was a protein that takes role in supplying nutrients (Grant, J. P., Ann. Surg., 220, 610-616 (1994)). Contrary to the functions of the tumor suppressor gene as reported previously, it was however found from this study result that the GIG20 tumor suppressor gene was not at all expressed in the liver cancer, while its expression was significantly increased in various normal liver tissues. The fact that the gene of the present invention is a tumor suppressor gene is based on that the protein “albumin” is produced in the normal liver cell because albumin genes within nucleuses are present in the liver cell. This is why that the normal liver cell is a cell in which the albumin gene normally works, but if a level of albumin is lower than the normal level in the liver cell, then the albumin gene does not normally works in the liver cell, or the number of the normal albumin gene is reduced, which may appear in the case of liver cancer

The DNA sequence of SEQ ID NO: 13 has one open reading frame (ORF) corresponding to base positions from 8 to 1261 of the DNA sequence (Base positions from 1259 to 1261 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 417 amino acid residues, and has an amino acid sequence of SEQ ID NO: 14 and a molecular weight of approximately 47 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 13. As another example, a 256-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP40 of SEQ ID NO: 15 (5′-AAGCTTGTCAGCC-3′) and an anchored oligo-dT primer of SEQ ID NO: 16 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a HepG2 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG20-cDNA into an expression vector pcDNA3.1 (Invitrogen, U.S.), and then transformed Escherichia coli DH5α with the resultant expression vector to obtain a transformant, which was then named E. coli DH5α/GIG20/pcDNA3.1, and deposited with Accession No. KCTC 10657BP into Korean Collection for Type Cultures on Jun. 14, 2004.

It is regarded that the gene of the present invention is overexpressed in normal tissues, preferably liver tissues, to suppress carcinogenesis. The gene of the present invention is mainly overexpressed in these tissues as an mRNA transcript having a size of approximately 2.4 kb. Especially, the gene of the present invention is differentially expressed only in the normal tissues. For example, the gene of the present invention is not expressed in the cancer tissues and the cancer cells such as the liver cancer tissue, the liver cancer cell line HepG2, etc., but differentially expressed only in the normal liver tissue.

The liver cancer cell line into which the genes of the present invention were introduced showed a high mortality, and therefore the gene of the present invention may be effectively used for treatment and prevention of the cancer.

5. GIG22

The gene of the present invention is a human cancer suppressor gene 22 (GIG22) having a DNA sequence of SEQ ID NO: 17, which has been deposited with Accession No. AY512565 into the GenBank database of U.S. National Institutes of Health (NIH) (Publication Date: May 31, 2005), and some DNA sequence of the deposited gene is different to a DNA sequence of the Homo sapiens DNAJ domain-containing protein MCJ gene deposited with Accession No. AF126743 into the database and its expressed amino acid sequence is also different to that of the Homo sapiens DNAJ domain-containing protein MCJ. It was reported that expression of the MCJ gene was reduced in the case of ovarian cancer (Shridhar, V. et al., Cancer Res., 61, 4258-4265 (2001)), but it was found from this study result that the GIG22 tumor suppressor gene was not at all expressed in various human tumors including liver cancer, while its expression was significantly increased in various normal tissues.

The DNA sequence of SEQ ID NO: 17 has one open reading frame (ORF) corresponding to base positions from 95 to 547 of the DNA sequence (Base positions from 545 to 547 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 150 amino acid residues, and has an amino acid sequence of SEQ ID NO: 18 and a molecular weight of approximately 16 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 17. As another example, a 281-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP30 of SEQ ID NO: 19 (5′-AAGCTTCGTACGT-3′) and an anchored oligo-dT primer of SEQ ID NO: 20 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a HepG2 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG22 cDNA into an expression vector pcDNA3.1 (Invitrogen, U.S.), and then transformed Escherichia coli DH5α with the resultant expression vector to obtain a transformant, which was then named E. coli DH5α/GIG22/pcDNA3.1, and deposited with Accession No. KCTC 10658BP into Korean Collection for Type Cultures on Jun. 14, 2004.

It is regarded that the gene of the present invention is overexpressed in normal tissues, preferably heart, muscles, liver, kidney, placenta, spleen, lungs, small and large intestines, spleen, thymus and leucocyte, to suppress carcinogenesis. It is regarded that the gene of the present invention is suppressed in leukemia, uterine cancer, malignant lymphoma, colon cancer, lung cancer and skin cancer to induce carcinogenesis. The gene of the present invention is mainly overexpressed in these tissues as an mRNA transcript having a size of approximately 0.6 kb. Especially, the gene of the present invention is differentially expressed only in the normal tissues. For example, the gene of the present invention is not expressed in the cancer tissues and the cancer cells such as the liver cancer tissue, the liver cancer cell line HepG2, etc., but differentially expressed only in the normal liver tissue.

The cancer cell line into which the genes of the present invention were introduced showed a high mortality, and therefore the gene of the present invention may be effectively used for treatment and prevention of the cancer.

6. GIG25

The gene of the present invention is a human cancer suppressor gene 25 (GIG25) having a DNA sequence of SEQ ID NO: 21, which has been deposited with Accession No. AY513276 into the GenBank database of U.S. National Institutes of Health (NIH) (Publication Date: Dec. 31, 2005), and some DNA sequence of the deposited gene is different to that of the Homo sapiens serine (or cysteine) proteinase inhibitor, clade A (alpha-1 antiproteinase, antitrypsin), member 3 gene deposited with Accession No. BC0110530 into the existing database. The alpha-1 antitrypsin is a typical member of serine (or cysteine) proteinase inhibitors, and it has been known that the alpha-1 antitrypsin was an acute-phase protein and its expression level was increased three to four times upon inflammatory reaction (Morgan, K., & Kalsherker, N. A., Int. J. Biochem. Cell Biol., 29, 1501-1511 (1997)). Contrary to the functions of the tumor suppressor gene as reported previously, it was however found from this study result that the GIG25 tumor suppressor gene was not at all expressed in the liver cancer, while its expression was significantly increased in various normal liver tissues.

The DNA sequence of SEQ ID NO: 21 has one open reading frame (ORF) corresponding to base positions from 436 to 1299 of the DNA sequence (Base positions from 434 to 436 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 287 amino acid residues, and has an amino acid sequence of SEQ ID NO: 22 and a molecular weight of approximately 33 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 21. As another example, a 250-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP40 of SEQ ID NO: 23 (5′-AAGCTTGTCAGCC-3′) and an anchored oligo-dT primer of SEQ ID NO: 24 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, a HepG2 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG25 cDNA into an expression vector pcDNA3.1 (Invitrogen, U.S.), and then transformed Escherichia coli DH5α with the resultant expression vector to obtain a transformant, which was then named E. coli DH5α/GIG25/pcDNA3.1, and deposited with Accession No. KCTC 10659BP into Korean Collection for Type Cultures on Jun. 14, 2004.

It is regarded that the gene of the present invention is overexpressed in normal tissues, preferably liver tissues, to suppress carcinogenesis. The genes of the present invention is mainly overexpressed in these tissues as an mRNA transcript having a size of approximately 1.5 kb. Especially, the gene of the present invention is differentially expressed only in the normal tissues. For example, the gene of the present invention is not expressed in the cancer tissues and the cancer cells such as the liver cancer tissue, the liver cancer cell line HepG2, etc., but differentially expressed only in the normal liver tissue.

The liver cancer cell line into which the genes of the present invention were introduced showed a high mortality, and therefore the gene of the present invention may be effectively used for treatment and prevention of the cancer.

7. GIG36

The gene of the present invention is a human cancer suppressor gene 36 (GIG36) having a DNA sequence of SEQ ID NO: 25, which has been deposited with Accession No. AY542304 into the GenBank database of U.S. National Institutes of Health (NIH) (Publication Date: Dec. 31, 2005), and the DNA sequence of the deposited gene is identical with that of the matrix Gla protein deposited with Accession No. M58549 into the existing database, and only one base pair of its DNA sequence is different to that of the matrix Gla protein deposited with Accession No. BC005272. It was reported that the matrix Gla protein was mainly secreted in vascular smooth muscle cells (Shanahan, C. M., et al., Crit. Rev. Eukaryot Gene Express., 8, 357-375 (1998)) and chondrocytes (Hale, J. E., et al., J. Biol. Chem., 263, 5820-5824 (1988)), and its function was to suppress mineralization (Luo, G., et al., Nature, 386, 78-81 (1997); Price, P. A., et al., Arterioscler. Thromb. Vasc. Biol., 18, 1400-1407 (1998); Price, P. A., et al., Arterioscler. Thromb. Vasc. Biol., 20, 317-327 (2000). It was also reported that expression of the matrix Gla gene was increased in some of cancers including ovarian cancer (Colleen, D., et al., Cancer Res., 61, 3869-3876 (2001)) and breast cancer (Chen, L., et al., Oncogene, 5, 1391-1395 (1990)). Contrary to the studies as reported previously, it was however found from this study result that the GIG36 tumor suppressor gene was not at all expressed in various human tumors including the liver cancer, while its expression was significantly increased in various normal tissues.

The DNA sequence of SEQ ID NO: 25 has one open reading frame (ORF) corresponding to base positions from 12 to 323 of the DNA sequence (Base positions from 321 to 323 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 103 amino acid residues, and has an amino acid sequence of SEQ ID NO: 26 and a molecular weight of approximately 12 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 25. As another example, a 182-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP29 of SEQ ID NO: 27 (5′-AAGCTTAGCAGCA-3′) and an anchored oligo-dT primer of SEQ ID NO: 28 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, an MCF-7 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG36 cDNA into an expression vector pcDNA3.1 (Invitrogen, U.S.), and then transformed Escherichia coli DH5α with the resultant expression vector to obtain a transformant, which was then named E. coli DH5α/GIG36/pcDNA3.1, and deposited with Accession No. KCTC 10643BP into Korean Collection for Type Cultures on May 24, 2004.

It is regarded that the gene of the present invention is overexpressed in normal tissues, preferably liver, kidney, spleen and lungs, to suppress carcinogenesis. It is also regarded that the gene of the present invention is suppressed even in leukemia, uterine cancer, malignant lymphoma, colon cancer and skin cancer to induce carcinogenesis. The gene of the present invention is mainly overexpressed in these tissues as an mRNA transcript having a size of approximately 1.3 kb. Especially, the gene of the present invention is differentially expressed only in the normal tissues. For example, the gene of the present invention is not expressed in the cancer tissues and the cancer cells such as the liver cancer tissue, the liver cancer cell line HepG2, etc., but differentially expressed only in the normal liver tissue.

The cancer cell line into which the genes of the present invention were introduced showed a high mortality, and therefore the gene of the present invention may be effectively used for treatment and prevention of the cancer.

8. GIG2

The gene of the present invention is a human cancer suppressor gene 2 (GIG2) having a DNA sequence of SEQ ID NO: 29, which has been deposited with Accession No. AY423720 into the GenBank database of U.S. National Institutes of Health (NIH) (Publication Date: Dec. 31, 2004), and the DNA sequence of the deposited gene is identical with those of the Homo sapiens mRNA for motility-related protein (MRP-1) gene and the Homo sapiens CD9 antigen (p24) (CD9) gene, deposited with Accession No. X60111 and NM_{—}001769 into the database, respectively. That is to say, it was reported that the Homo sapiens mRNA for motility-related protein (MRP-1) gene deposited with Accession No. X60111 was associated with cell migration (Miyake, M., et al., J. Exp. Med., 174, 1347-1354 (1991)). It was also reported that the Homo sapiens CD9 antigen (p24) (CD9) gene deposited with Accession No. NM_{—}001769 was associated with cell migration and an invasive ability in the breast cancer (Sauer, G. et al., Oncol. Rep., 10, 405-410 (2003)) and the lung cancer (Funakoshi, T. et al., Oncogene, 22, 674-687 (2003)).

Contrary to the cell migration and the invasive ability as reported previously, it was however found from this study result that the GIG2 tumor suppressor gene was very slightly expressed or not at all expressed in various human tumors including the lung cancer, while its expression was significantly increased in various normal tissues.

The DNA sequence of SEQ ID NO: 29 has one open reading frame (ORF) corresponding to base positions from 18 to 704 of the DNA sequence (Base positions from 702 to 704 represent a stop codon). However, because of degeneracy of codons, or considering preference of codons for living organisms to express the genes, the genes of the present invention may be variously modified in coding regions without changing an amino acid sequence of the protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

The protein expressed from the gene of the present invention consists of 228 amino acid residues, and has an amino acid sequence of SEQ ID NO: 30 and a molecular weight of approximately 25 kDa. However, one or more amino acids may be also substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The gene and protein of the present invention may be separated from human tissues, or be synthesized according to the known methods for synthesizing DNA or peptide. For example, the gene of the present invention may be screened and cloned according to the conventional methods on the basis of the information on the DNA sequence set forth in SEQ ID NO: 29. As another example, a 240-bp cDNA fragment, which is not expressed in the cancer tissue or the cancer cell line but differentially expressed only in the normal tissue, may be obtained by carrying out a reverse transcription-polymerase chain reaction (RT-PCR) on the total RNAs extracted from a normal tissue, and a cancer tissue or a cancer cell line using a random primer H-AP32 of SEQ ID NO: 31 (5′-AAGCTTCTTGCAA-3′) and an anchored oligo-dT primer of SEQ ID NO: 32 (5′-AAGCTTTTTTTTTTTC-3′), and the resultant fragment, which is used as the probe, may be plaque-hybridized with a cDNA library to obtain a full-length cDNA clone.

The gene prepared thus may be inserted into a vector for expression in microorganisms or animal cells, already known in the art, to obtain an expression vector, and then cDNA of the gene may be replicated in a large quantity or its protein may be produced in a commercial quantity by introducing the expression vector into suitable host cells, for example Escherichia coli, an A549 cell line, etc. Upon constructing the expression vector, DNA regulatory sequences such as a promoter and a terminator, autonomously replicating sequences, secretion signals, etc. may be suitably selected and combined depending on kinds of the host cells that are engineered to produce the gene or the protein.

The present inventors inserted the full-length GIG2 cDNA into an expression vector pcDNA3.1 (Invitrogen, U.S.), and then transformed Escherichia coli DH5α with the resultant expression vector to obtain a transformant. which was then named E. coli DH5α/GIG2/pcDNA3.1, and deposited with Accession No. KCTC 10641BP into Korean Collection for Type Cultures on May 31, 2004.

It is regarded that the gene of the present invention is overexpressed in normal tissues, preferably brain, heart, muscles, large intestines, thymus, spleen, kidney, liver, small intestines, placenta, lungs and leucocyte, to suppress carcinogenesis. It is also regarded that the gene of the present invention is not at all expressed in acute leukemia (HL-60 cell line) and malignant lymphoma (the RaJi cancer cell line) to induce the cancer, and also slightly expressed in the uterine cancer, the chronic leukemia, the colon cancer, the lung cancer and the skin cancer to induce carcinogenesis. The gene of the present invention is mainly overexpressed in these tissues as an mRNA transcript having a size of approximately 1.3 kb. Especially, the gene of the present invention is differentially expressed only in the normal tissues. For example, the gene of the present invention is not expressed in the cancer tissues and the cancer cells such as the lung cancer tissue, the metastatic lung cancer tissue, the lung cancer cell line (A549 and NCI-H358), etc., but differentially expressed only in the normal lung tissue.

The cancer cell line into which the genes of the present invention were introduced showed a high mortality, and therefore the gene of the present invention may be effectively used for treatment and prevention of the cancer.

### MODE FOR INVENTION

Hereinafter, the present invention will be described in detail referring to preferred examples. Therefore, the description proposed herein is just a preferable example for the purpose of illustrations only, not intended to limit the scope of the invention.

### Reference Example

### Separation of Total RNA

The total RNA samples were separated from fresh tissues or cultured cells using the RNeasy total RNA kit (Qiagen Inc., Germany), and then the contaminated DNA was removed from the RNA samples using the message clean kit (GenHunter Corp., MA, U.S.).

### Example 1

### Separation of Total RNA and mRNA Differential Display

1-1. GIG12

A differential expression pattern of the gene of interest was measured in a normal breast tissue, a primary breast cancer tissue and a breast cancer cell line, as follows.

A normal breast tissue sample was obtained from a breast cancer patient during mastectomy surgery, and a primary breast cancer tissue sample was obtained during radical mastectomy surgery from a patient who did not undergo radiation or anti-cancer therapy before surgical treatment. MCF-7 (American Type Culture Collection; ATCC Number HTB-22) was used as the human breast cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 4 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP32 (RNAimage primer set 1, GenHunter Corporation, U.S.) of SEQ ID NO: 3. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 1 shows a PCR result using a 5′13-mer random primer H-AP32 of SEQ ID NO: 3 and an anchored oligo-dT primer of SEQ ID NO: 4. In FIG. 1, Lanes 1, 2 and 3 represent the normal breast tissues; Lanes 4, 5 and 6 represent the breast cancer tissues; and Lane 7 represents the breast cancer cell line MCF-7. As seen in FIG. 1, it was confirmed that a 680-bp cDNA fragment was not expressed in the breast cancer tissue and the breast cancer cell line, but differentially expressed only in the normal breast tissue (Base positions from 1614 to 2283 of the full-length GIG12 gene sequence). The cDNA fragment was named FC26.

A 680-bp band, FC5 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment FC26 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, its DNA sequence was identical with that of the matrix Gla protein deposited with Accession No. M58549 and BC005272 into the database.

1-2. GIG17

A differential expression pattern of the gene of interest was measured in a normal liver tissue, a primary liver cancer tissue and a liver cancer cell line, as follows.

Samples of a normal liver tissue and a liver cancer tissue were obtained from a liver cancer patient during tissue biopsy, and HepG2 (American Type Culture Collection; ATCC Number HB-8065) was used as the human liver cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 8 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP7 (RNAimage primer set 1, GenHunter Corporation, U.S.) of SEQ ID NO: 7. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

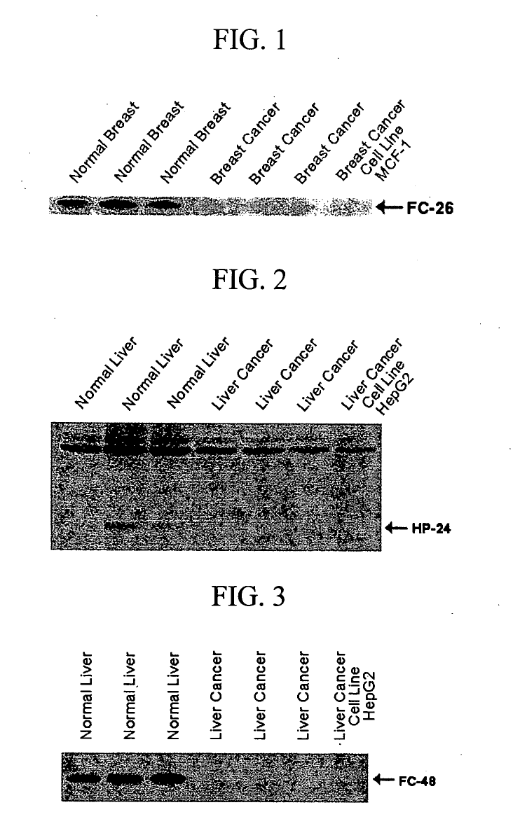

FIG. 2 shows a PCR result using a 5′13-mer random primer H-AP7 of SEQ ID NO: 7 and an anchored oligo-dT primer of SEQ ID NO: 8. In FIG. 1, Lanes 1, 2 and 3 represent the normal liver tissues; Lanes 4, 5 and 6 represent the liver cancer tissues; and Lane 7 represents the liver cancer cell line HepG2. As seen in FIG. 1, it was confirmed that a 250-bp cDNA fragment was very slightly expressed in the liver cancer tissue, not expressed in the liver cancer cell line, and differentially expressed only in the normal liver tissue (Base positions from 721 to 970 of the full-length GIG17 gene sequence). The cDNA fragment was named HP24.

A 250-bp band, HP24 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment FC5 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, its DNA sequence was identical with that of the human fructose 1,6-bisphosphatase deposited with Accession No. M19922 into the database.

1-3. GIG19

A differential expression pattern of the gene of interest was measured in a normal liver tissue, a primary liver cancer tissue and a liver cancer cell line, as follows.

Samples of a normal liver tissue and a liver cancer tissue were obtained from a liver cancer patient during tissue biopsy, and HepG2 (American Type Culture Collection; ATCC Number HB-8065) was used as the human liver cancer cell line. This experiment was repeated in the same manner as in the reference example to separate the total RNAs from these tissues and cells, respectively.

An RT-PCR reaction was carried out using each of the total RNA samples separated from the tissue and the cell according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 12 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP40 (RNAimage primer set 1, GenHunter Corporation, U.S.) of SEQ ID NO: 11. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 3 shows a PCR result using a 5′13-mer random primer H-AP40 of SEQ ID NO: 11 and an anchored oligo-dT primer of SEQ ID NO: 12. In FIG. 3, Lanes 1, 2 and 3 represent the normal liver tissues; Lanes 4, 5 and 6 represent the liver cancer tissues; and Lane 7 represents the liver cancer cell line HepG2. As seen in FIG. 3, it was confirmed that a 281-bp cDNA fragment was not expressed in the liver cancer tissue and the liver cancer cell line, but differentially expressed only in the normal liver tissue (Base positions from 781 to 1061 of the full-length GIG19 gene sequence). The cDNA fragment was named HP48.

A 281-bp band, HP48 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment FC5 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, its DNA sequence was identical with those of the Homo sapiens alpha-1-microglobulin/bikunin precursor and the human mRNA for protein HC (alpha-1-microglobulin), deposited with Accession No. BC041593 and X04225 into the database, respectively.

1-4. GIG20

A differential expression pattern of the gene of interest was measured in a normal liver tissue, a primary liver cancer tissue and a liver cancer cell line, as follows.

Samples of a normal liver tissue and a liver, cancer tissue were obtained from a liver cancer patient during tissue biopsy, and HepG2 (American Type Culture Collection; ATCC Number HB-8065) was used as the human liver cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 16 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP40 (RNAimage primer set 1, GenHunter Corporation, U.S.) of SEQ ID NO: 15. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

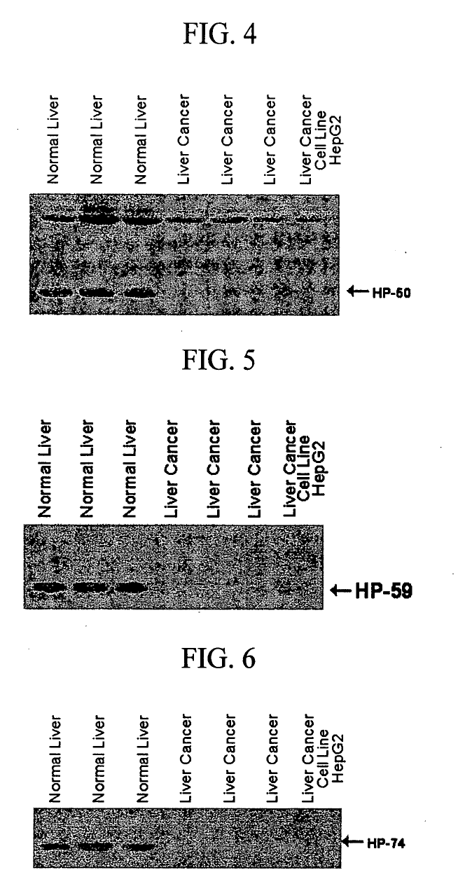

FIG. 4 shows a PCR result using a 5′13-mer random primer H-AP40 of SEQ ID NO: 15 and an anchored oligo-dT primer of SEQ ID NO: 16. In FIG. 4, Lanes 1, 2 and 3 represent the normal liver tissues; Lanes 4, 5 and 6 represent the liver cancer tissues; and Lane 7 represents the liver cancer cell line HepG2. As seen in FIG. 4, it was confirmed that a 256-bp cDNA fragment was not expressed in the liver cancer tissue and the liver cancer cell line, but differentially expressed only in the normal liver tissue (Base positions from 776 to 1031 of the full-length GIG19 gene sequence). The cDNA fragment was named HP50.

A 256-bp band, HP50 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment HP50 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, its DNA sequence was identical with that of the Homo sapiens albumin deposited with Accession No. BC041789 into the database.

1-5. GIG22

A differential expression pattern of the gene of interest was measured in a normal liver tissue, a primary liver cancer tissue and a liver cancer cell line, as follows.

Samples of a normal liver tissue and a liver cancer tissue were obtained from a liver cancer patient during tissue biopsy, and HepG2 (American Type Culture Collection; ATCC Number HB-8065) was used as the human liver cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 20 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP30 (RNAimage primer set 1, GenHunter Corporation, U.S.) of SEQ ID NO: 19. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 5 shows a PCR result using a 5′13-mer random primer H-AP30 of SEQ ID NO: 19 and an anchored oligo-dT primer of SEQ ID NO: 20. In FIG. 5, Lanes 1, 2 and 3 represent the normal liver tissues; Lanes 4, 5 and 6 represent the liver cancer tissues; and Lane 7 represents the liver cancer cell line HepG2. As seen in FIG. 5, it was confirmed that a 281-bp cDNA fragment was not expressed in the liver cancer tissue and the liver cancer cell line, but differentially expressed only in the normal liver tissue (Base positions from 262 to 542 of the full-length GIG22 gene sequence). The cDNA fragment was named HP59.

A 281-bp band, HP59 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment HP59 was cloned into an expression vector pGEM-T Easy using the TA cloning system (Promega), and then sequenced using the Sequenase Version 2.0 DNA Sequencing System (United States Biochemical Co.).

1-6. GIG25

A differential expression pattern of the gene of interest was measured in a normal liver tissue, a primary liver cancer tissue and a liver cancer cell line, as follows.

Samples of a normal liver tissue and a liver cancer tissue were obtained from a liver cancer patient during tissue biopsy, and HepG2 (American Type Culture Collection; ATCC Number HB-8065) was used as the human liver cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 24 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-AP40 (RNAimage primer set 1, GenHunter Corporation, U.S.) of SEQ ID NO: 23. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 6 shows a PCR result using a 5′13-mer random primer H-AP40 of SEQ ID NO: 23 and an anchored oligo-dT primer of SEQ ID NO: 24. In FIG. 1, Lanes 1, 2 and 3 represent the normal liver tissues; Lanes 4, 5 and 6 represent the liver cancer tissues; and Lane 7 represents the liver cancer cell line HepG2. As seen in FIG. 6, it was confirmed that a 250-bp cDNA fragment was not expressed in the liver cancer tissue and the liver cancer cell line, but differentially expressed only in the normal liver tissue (Base positions from 1201 to 1450 of the full-length GIG25 gene sequence). The cDNA fragment was named HP74.

A 250-bp band, HP74 fragment, was remove from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment HP74 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, some of its DNA sequence was different to that of the Homo sapiens serine (or cysteine) proteinase inhibitor, clade A (alpha-1 antiproteinase, antitrypsin), member 3 deposited with Accession No. BC0110530 into the database.

1-7. GIG36

A differential expression pattern of the gene of interest was measured in a normal breast tissue, a primary breast cancer tissue and a breast cancer cell line, as follows.

A normal breast tissue sample was obtained from a breast cancer patient during mastectomy surgery, and a primary breast cancer tissue sample was obtained during radical mastectomy from a patient who did not undergo radiation or anti-cancer therapy before surgical treatment. MCF-7 (American Type Culture Collection; ATCC Number HTB-22) was used as the human breast cancer cell line. This experiment was repeated in the same manner as in the reference example to separate the total RNAs from these tissues and cells, respectively.

An RT-PCR reaction was carried out using each of the total RNA samples separated from the tissue and the cell according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 28 using a kit (a RNAimage kit, GenHunter), and then a PCR reaction was carried out in the presence of 0.5 mM [(α-³⁵S]-labeled dATP (1,200 Ci/mmol) using the same anchored oligo-dT primer and a 5′13-mer random primer H-AP29 (RNAimage primer set 1, GenHunter Corporation, U.S.) of SEQ ID NO: 27. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

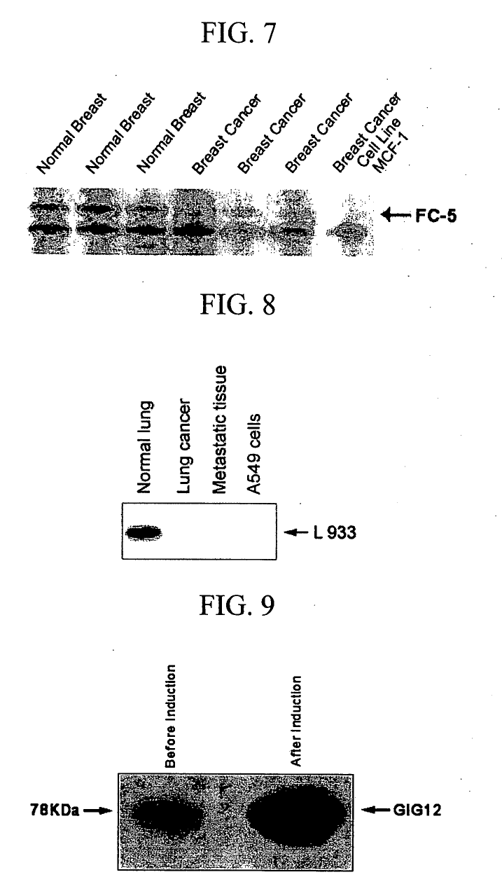

FIG. 7 shows a PCR result using a 5′13-mer random primer H-AP29 of SEQ ID NO: 27 and an anchored oligo-dT primer of SEQ ID NO: 28. In FIG. 1, Lanes 1, 2 and 3 represent the normal breast tissues; Lanes 4, 5 and 6 represent the breast cancer tissues; and Lane 7 represents the breast cancer cell line MCF-7. As seen in FIG. 7, it was confirmed that a 182-bp cDNA fragment was not expressed in the breast cancer tissue and the breast cancer cell line, but differentially expressed only in the normal breast tissue (Base positions from 183 to 364 of the full-length GIG36 gene sequence). The cDNA fragment was named FC5.

A 182-bp band, FC5 fragment, was removed from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment FC5 was cloned into an expression vector pGEM-T Easy using the TA cloning system (Promega), and then sequenced using the Sequenase Version 2.0 DNA Sequencing System (United States Biochemical Co.). This DNA sequence was searched in the GenBank database of U.S. National Institutes of Health (NIH) using the BLAST and FASTA program. As a result, its DNA sequence was identical with those of the matrix Gla proteins, deposited with Accession No. M58549 and BC005272 into the database, respectively.

1-8. GIG2

A differential expression pattern of the gene of interest was measured in a normal lung tissue, a primary lung cancer tissue, a metastatic lung cancer tissue and a lung cancer cell line, as follows. Samples of a normal lung tissue, a lung cancer tissue and a metastatic lung cancer tissue were obtained from a lung cancer patient during surgery, and A549 (American Type Culture Collection; ATCC Number CCL-185) and NCI-H358 (American Type Culture Collection; ATCC Number CRL-5807) were used as the human lung cancer cell line. This experiment was repeated in the same manner as in the reference example to separate the total RNAs from these tissues and cells, respectively.

An RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 32 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a 5′13-mer random primer H-A32 (RNAimage primer set 1, GenHunter Corporation, U.S.) of SEQ ID NO: 31. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 8 shows a PCR result using a 5′13-mer random primer H-AP32 of SEQ ID NO: 31 and an anchored oligo-dT primer of SEQ ID NO: 32. In FIG. 8, Lanes 1, 2 and 3 represent the normal lung tissues; Lanes 4, 5 and 6 represent the lung cancer tissues; and Lane 7 represents the lung cancer cell line NCI-H358. As seen in FIG. 8, it was confirmed that a 240-bp cDNA fragment was not expressed in the lung cancer tissue, the metastatic lung cancer tissue and the lung cancer cell line, but differentially expressed only in the normal lung tissue (Base positions from 371 to 610 of the full-length GIG2 gene sequence). The cDNA fragment was named L933.

A 240-bp band, L933 fragment, was remove from the dried gell, boiled for 15 minutes to elute cDNA, and a PCR reaction was then carried out under the same said condition using the same said primer set to re-amplify the cDNA, except that the [α-³⁵S]-labeled dATP and the 20 μM dNTP were not used herein. The re-amplified cDNA fragment L933 was cloned into an expression vector pGEM-T Easy using the TA cloning system (Promega), and then sequenced using the Sequenase Version 2.0 DNA Sequencing System (United States Biochemical Co.).

### Example 2

### cDNA Library Screening

The cDNA fragments FC26 obtained in Example 1-1, HP24 obtained in Example 1-2, HP48 obtained in Example 1-3, HP50 obtained in Example 1-4, HP59 obtained in Example 1-5, HP74 obtained in Example 1-6, FC5 obtained in Example 1-7 and L933 obtained in Example 1-8 were labeled according to the method of the disclosure (Feinberg, A. P. and Vogelstein, B., Anal. Biochem., 132, 6-13 (1983)), respectively, to obtain ³²P-labeled cDNA probes, and the resultant ³²P-labeled cDNA probes was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory manual, New York: Cold Spring Harbor Laboratory (1989)) to obtain full-length cDNA clones of the human cancer suppressor genes GIG12, GIG17, GIG19, GIG20, GIG22, GIG25, GIG36 and GIG2, respectively.

The full-length cDNAs were sequenced, and therefore their DNA sequences were identical with SEQ ID NO: 1 (GIG12), SEQ ID NO: 5 (GIG17), SEQ ID NO: 9 (GIG19), SEQ ID NO: 13 (GIG20), SEQ ID NO: 17 (GIG22), SEQ ID NO: 21 (GIG25), SEQ ID NO: 25 (GIG36) and SEQ ID NO: 29 (GIG2), respectively.

The GIG12 sequence has an open reading frame encoding 711 amino acid residues, and the amino acid sequence derived from the open reading frame was identical with SEQ ID NO: 2. The derived protein also had a molecular weight of approximately 78 kDa.

The GIG17 sequence also has an open reading frame encoding 388 amino acid residues, and the amino acid sequence derived from the open reading frame was identical with SEQ ID NO: 6. The derived protein also had a molecular weight of approximately 37 kDa.

The GIG19 sequence also has an open reading frame encoding 352 amino acid residues, and the amino acid sequence derived from the open reading frame was identical with SEQ ID NO: 10. The derived protein also had a molecular weight of approximately 39 kDa.

The GIG20 sequence also has an open reading frame encoding 417 amino acid residues, and the amino acid sequence derived from the open reading frame was identical with SEQ ID NO: 14. The derived protein also had a molecular weight of approximately 47 kDa.

The GIG22 sequence also has an open reading frame encoding 150 amino acid residues, and the amino acid sequence derived from the open reading frame was identical with SEQ ID NO: 18. The derived protein also had a molecular weight of approximately 16 kDa.

The GIG25 sequence also has an open reading frame encoding 287 amino acid residues, and the amino acid sequence derived from the open reading frame was identical with SEQ ID NO: 22. The derived protein also had a molecular weight of approximately 33 kDa.

The GIG36 sequence also has an open reading frame encoding 103 amino acid residues, and the amino acid sequence derived from the open reading frame was identical with SEQ ID NO: 26. The derived protein also had a molecular weight of approximately 12 kDa.

The GIG2 sequence also has an open reading frame encoding 228 amino acid residues, and the amino acid sequence derived from the open reading frame was identical with SEQ ID NO: 30. The derived protein also had a molecular weight of approximately 25 kDa.

Each of the resultant full-length GIG cDNA clones was inserted into a eukaryotic expression vector pcDNA3.1 (Invitrogen, U.S.) to obtain eukaryotic expression vectors, and Escherichia coli DH5α was then transformed with the resultant eukaryotic expression vectors to obtain transformants. Each of the transformants was named E. coli DH5α/GIG12/pcDNA3.1, and then deposited Accession No. KCTC 10642BP (GIG12) into Korean Collection for Type Cultures on May 24, 2004; the resultant transformant was named E. coli DH5α/GIG17/pcDNA3.1, and then deposited with Accession No. KCTC 10655BP (GIG17) into Korean Collection for Type Cultures on Jun. 14, 2004; the resultant transformant was named E. coli DH5α/GIG19/pcDNA3.1, and then deposited with Accession No. KCTC 10656BP (GIG19) into Korean Collection for Type Cultures on Jun. 14, 2004; the resultant transformant was named E. coli DH5α/GIG20/pcDNA3.1, and then deposited with Accession No. KCTC 10657BP (GIG20) into Korean Collection for Type Cultures on Jun. 14, 2004; the resultant transformant was named E. coli DH5α/GIG22/pcDNA3.1, and then deposited with Accession No. KCTC 10658BP (GIG22) into Korean Collection for Type Cultures on Jun. 14, 2004; the resultant transformant was named E. coli DH5α/GIG25/pcDNA3.1, and then deposited with Accession No. KCTC 10659BP (GIG25) into Korean Collection for Type Cultures on Jun. 14, 2004; the resultant transformant was named E. coli DH5α/GIG36/pcDNA3.1, and then deposited with Accession No. KCTC 10643BP (GIG36) into Korean Collection for Type Cultures on May 24, 2004; and the full-length GIG2 cDNA clone was inserted into a eukaryotic expression vector pcDNA3.1 (Invitrogen, U.S.), and then Escherichia coli DH5α was transformed by the resultant eukaryotic expression vectors, and the resultant transformant was named E. coli DH5α/GIG2/pcDNA3.1, and then deposited with Accession No. KCTC 10641BP (GIG2) into Korean Collection for Type Cultures on May 31, 2004.

The transformed E. coli strain was culture in LB broth, and 0.2 M L-arabinose (Sigma, U.S.) was added to the culture media, and then reacted at 37° C. for 3 hours to express the GIG36 gene. Protein samples was obtained from the resultant culture media, and then SDS-PAGE was conducted with the resultant protein samples according to the method as described in the disclosure (Sambrook, J. et al., Molecular Cloning: A Laboratory manual, New York: Cold Spring Harbor Laboratory (1989)).

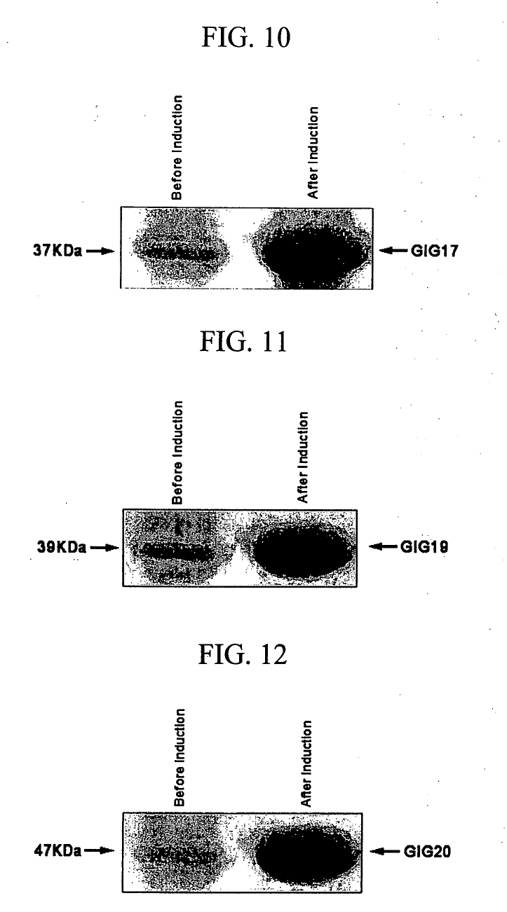

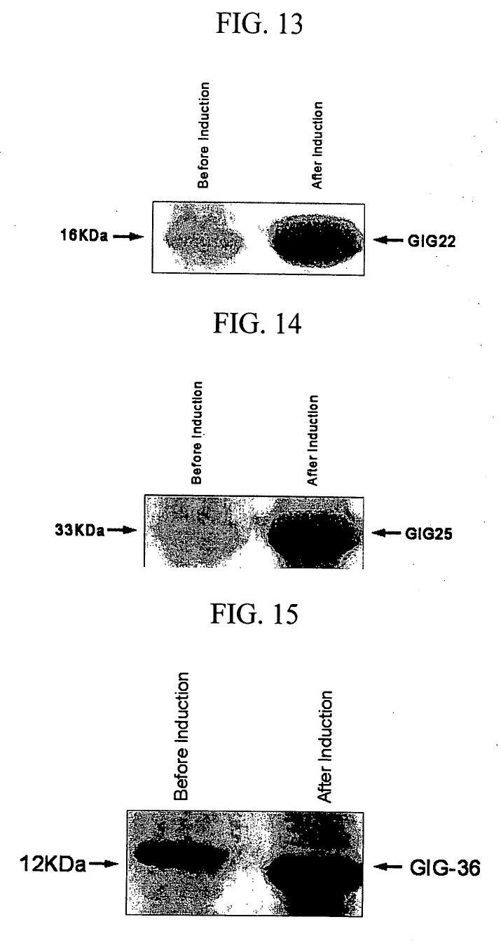

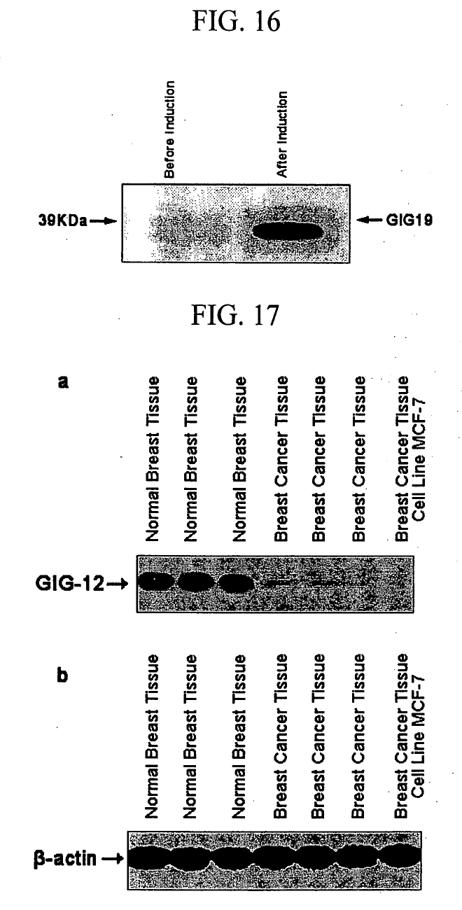

FIGS. 9, 10, 11, 12, 13, 14, 15 and 16 show results that the gene products of GIG12, GIG17, GIG19, GIG20, GIG22, GIG25, GIG36 and GIG2 are analyzed on SDS-PAGE, respectively. In FIG. 2, Lane 1 represents the protein sample before IPTG induction, and Lane 2 represents the protein sample after expression of the GIG gene is induced by IPTG, respectively. As shown in FIG. 2, the expressed GIG12 protein has a molecular weight of approximately 78 kDa, which corresponds to the molecular weight derived from its DNA sequence; the expressed GIG17 protein has a molecular weight of approximately 37 kDa, which corresponds to the molecular weight derived from its DNA sequence; the expressed GIG19 protein has a molecular weight of approximately 39 kDa, which corresponds to the molecular weight derived from its DNA sequence; the expressed GIG20 protein has a molecular weight of approximately 47 kDa, which corresponds to the molecular weight derived from its DNA sequence; the expressed GIG22 protein has a molecular weight of approximately 16 kDa, which corresponds to the molecular weight derived from its DNA sequence; the expressed GIG25 protein has a molecular weight of approximately 33 kDa, which corresponds to the molecular weight derived from its DNA sequence; the expressed GIG36 protein has a molecular weight of approximately 12 kDa, which corresponds to the molecular weight derived from its DNA sequence; and the expressed GIG2 protein has a molecular weight of approximately 25 kDa, which corresponds to the molecular weight derived from its DNA sequence.

### Example 3

### Northern Blotting of GIG Gene

3-1. Northern Blotting of GIG12 Gene

In order to assess an expression level of the GIG12 gene, the northern blotting was carried out, as follows.

20 μg of each of the total RNA samples obtained from the three normal breast tissues, the three primary breast cancer tissues and the breast cancer cell line MCF-7 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from a partial sequence FC26 cDNA of the full-length GIG12 cDNA using the Rediprime II random prime labelling system (Amersham, United Kingdom). The northern blotting procedure was repeated twice; one was quantified using the densitometer and the other was hybridized with the β-actin probe to determine the total mRNA.

FIG. 17(a) shows the northern blotting result that the GIG12 gene is differentially expressed in a normal breast tissue, a primary breast cancer tissue and a breast cancer cell line, and FIG. 17(b) shows the northern blotting result obtained by hybridizing the same blot with β-actin probe. As shown in FIGS. 17(a) and (b), it was revealed that the expression level of the GIG12 gene was highly detected all in the three samples of the normal breast tissue, but its expression level was significantly lower in the three samples of the breast cancer tissue than the normal tissue, and not detected in the one sample of the breast cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (RaJi), SW480 colon cancer cell, A549 lung cancer cell and G361 melanoma cell.

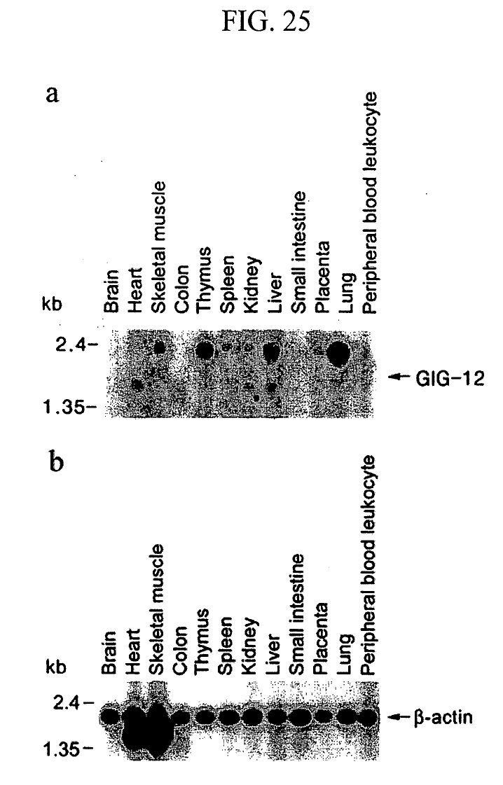

FIG. 25(a) shows a northern blotting result that the GIG12 gene is differentially expressed in various normal tissues, and FIG. 25(b) shows a northern blotting result obtained by hybridizing the same blot with β-actin probe. As shown in FIG. 25(a), dominant GIG12 mRNA transcript having a size of approximately 2.4 kb was overexpressed in the normal tissues such as lungs, thymus, liver, skeletal muscles, kidney, spleen, heart, placenta and peripheral blood.

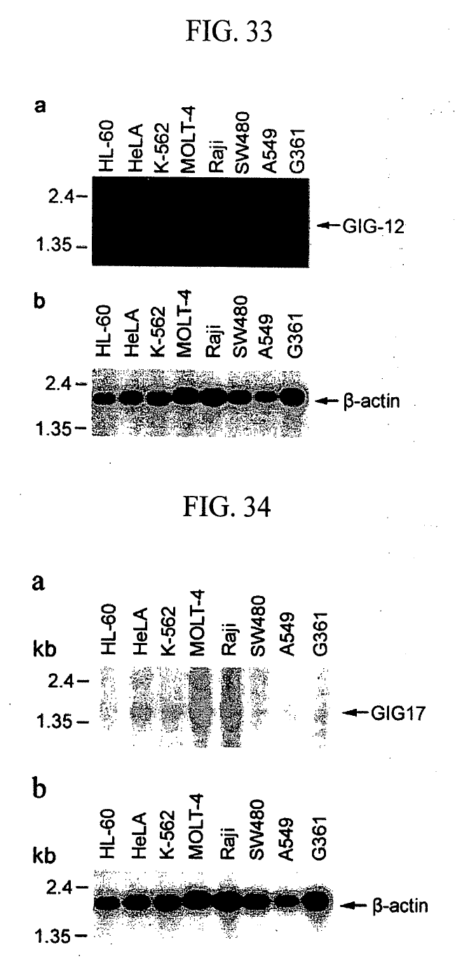

FIG. 33(a) shows a northern blotting result that the GIG12 gene is differentially expressed in various cancer cell lines, and FIG. 33(b) shows a northern blotting result obtained by hybridizing the same blot with β-actin probe. As shown in FIG. 33(a), the GIG12 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG12 gene of the present invention had the tumor suppresser function in the normal tissues such as breast, lungs, thymus, liver, skeletal muscles, kidney, spleen, heart, placenta, and peripheral blood.

3-2. Northern Blotting of GIG17 Gene

In order to assess an expression level of the GIG17 gene, the northern blotting was carried out, as follows.

20 μg of each of the total RNA samples obtained from the three normal liver tissues, the three primary liver cancer tissues and the liver cancer cell line HepG2 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG17 cDNA using the Rediprime II random prime labelling system (Amersham, United Kingdom). The northern blotting procedure was repeated twice; one was quantified using the densitometer and the other was hybridized with the β-actin probe to determine the total mRNA.

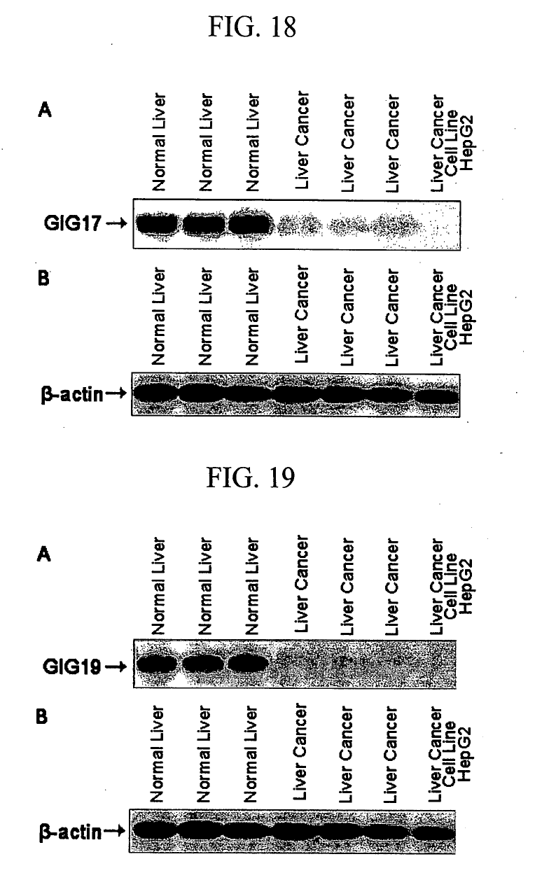

FIG. 18(a) shows the northern blotting result that the GIG17 gene is differentially expressed in a normal liver tissue, a primary liver cancer tissue and a liver cancer cell line, and FIG. 18(b) shows the northern blotting result obtained by hybridizing the same blot with β-actin probe. As shown in FIGS. 18(a) and (b), it was revealed that the expression level of the GIG17 gene was highly detected all in the three samples of the normal liver tissue, but its expression level was significantly lower in the three samples of the liver cancer tissue than the normal tissue, and not detected in the one sample of the liver cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines were transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

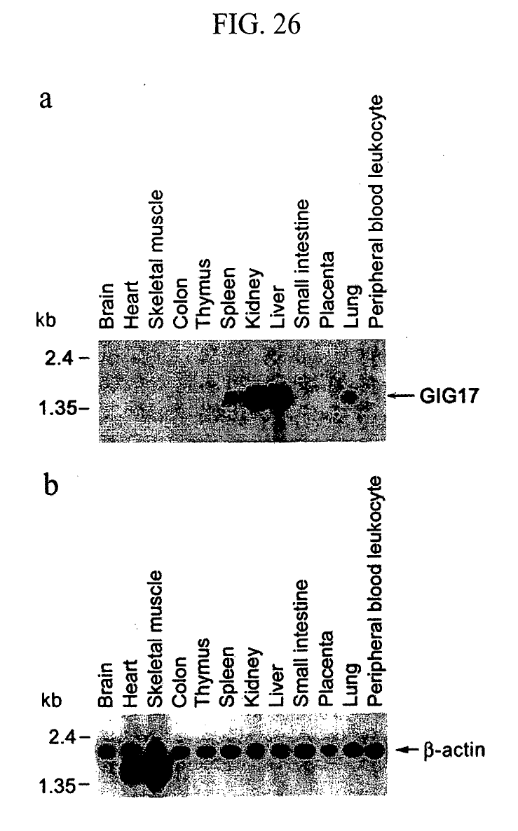

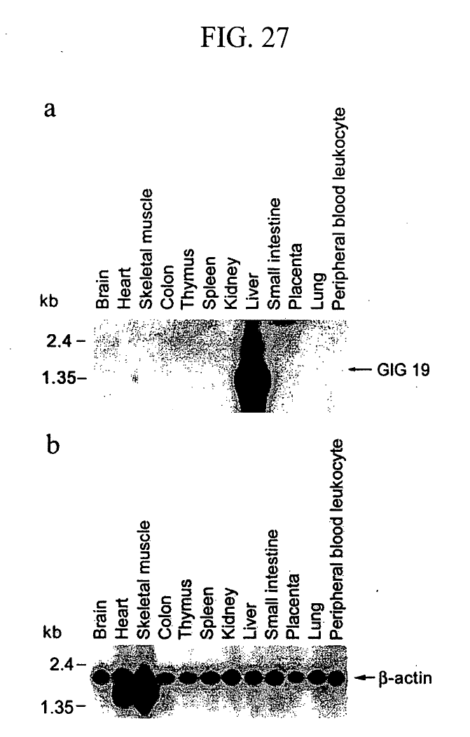

FIG. 26(a) shows a northern blotting result that the GIG17 gene is differentially expressed in various normal tissues, and FIG. 26(b) shows a northern blotting result obtained by hybridizing the same blot with β-actin probe. As shown in FIG. 26(a), dominant GIG17 mRNA transcript having a size of approximately 1.7 kb was overexpressed in the normal tissues such as liver, kidney, spleen and lungs.

FIG. 34(a) shows a northern blotting result that the GIG17 gene is differentially expressed in various cancer cell lines, and FIG. 34(b) shows a northern blotting result obtained by hybridizing the same blot with β-actin probe. As shown in FIG. 34(a), the GIG17 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG17 gene of the present invention had the tumor suppresser function in the normal tissues such as liver, kidney, spleen and lungs. Also, it was revealed that the GIG17 gene of the present invention had the tumor suppresser function, considering that its expression was suppressed in the tissues such as leukemia, uterine cancer, malignant lymphoma, colon cancer, skin cancer, etc. to induce carcinogenesis.

3-3. Northern Blotting of GIG19 Gene

In order to assess an expression level of the GIG19 gene, the northern blotting was carried out, as follows.

20 μg of each of the total RNA samples obtained from the three normal liver tissues, the three primary liver cancer tissues and the liver cancer cell line HepG2 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the HP48 cDNA using the Rediprime II random prime labelling system (Amersham, United Kingdom). The northern blotting procedure was repeated twice; one was quantified using the densitometer and the other was hybridized with the β-actin probe to determine the total mRNA.

FIG. 19(a) shows the northern blotting result that the GIG19 gene is differentially expressed in a normal liver tissue, a primary liver cancer tissue and a liver cancer cell line, and FIG. 19(b) shows the northern blotting result obtained by hybridizing the same blot with β-actin probe. As shown in FIGS. 19(a) and (b), it was revealed that the expression level of the GIG19 gene was highly detected all in the three samples of the normal liver tissue, but its expression level was not detected in the three samples of the liver cancer tissue and the one sample of the liver cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

FIG. 27(a) shows a northern blotting result that the GIG19 gene is differentially expressed in various normal tissues, and FIG. 27(b) shows a northern blotting result obtained by hybridizing the same blot with β-actin probe. As shown in FIG. 27(a), dominant GIG19 mRNA transcript having a size of approximately 1.2 kb was overexpressed only in the normal liver tissue.

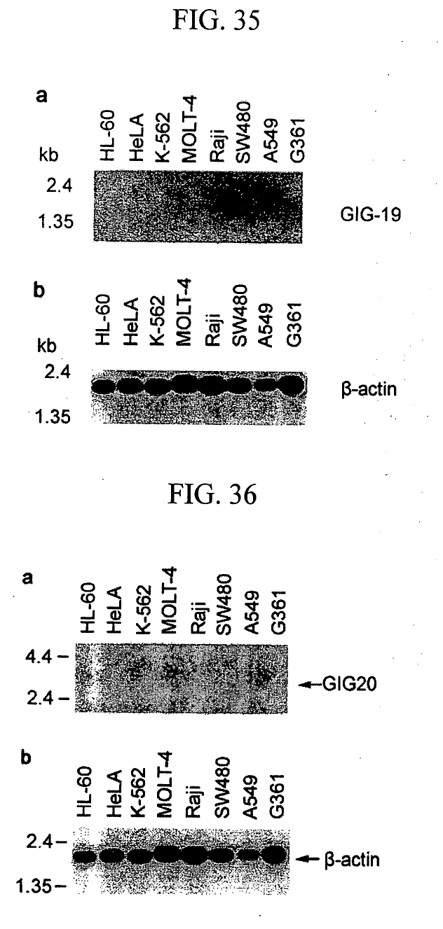

FIG. 35(a) shows a northern blotting result that the GIG19 gene is differentially expressed in various cancer cell lines, and FIG. 35(b) shows a northern blotting result obtained by hybridizing the same blot with β-actin probe. As shown in FIG. 35(a), the GIG19 gene was not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG19 gene of the present invention had the tumor suppresser function in the normal liver tissue.

3-4. Northern Blotting of GIG20 Gene

In order to assess an expression level of the GIG20 gene, the northern blotting was carried out, as follows.

20 μg of each of the total RNA samples obtained from the three normal liver tissues, the three primary liver cancer tissues and the liver cancer cell line HepG2 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the HP50 cDNA using the Rediprime II random prime labelling system (Amersham, United Kingdom). The northern blotting procedure was repeated twice; one was quantified using the densitometer and the other was hybridized with the β-actin probe to determine the total mRNA.

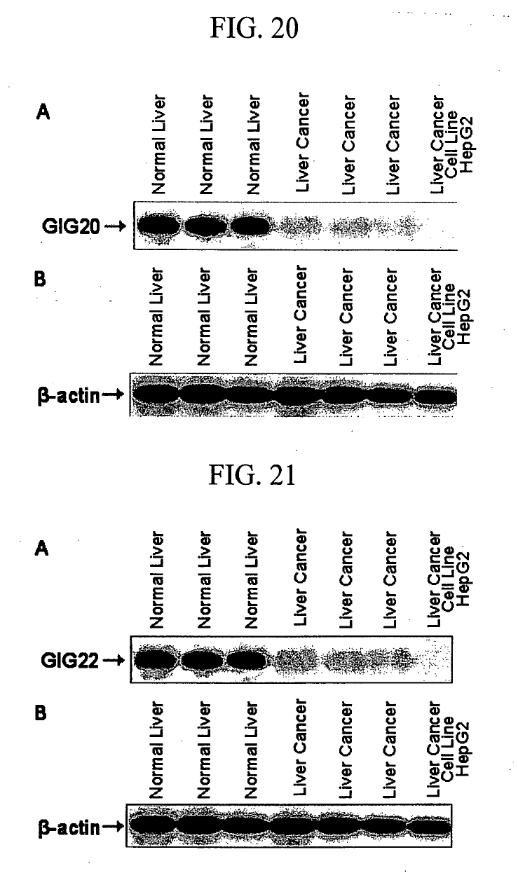

FIG. 20(a) shows the northern blotting result that the GIG20 gene is differentially expressed in a normal liver tissue, a primary liver cancer tissue and a liver cancer cell line, and FIG. 20(b) shows the northern blotting result obtained by hybridizing the same blot with β-actin probe. As shown in FIGS. 20(a) and (b), it was revealed that the expression level of the GIG20 gene was highly detected all in the samples of the three normal liver tissue, but its expression level was not detected in the three samples of the liver cancer tissue and the one sample of the liver cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

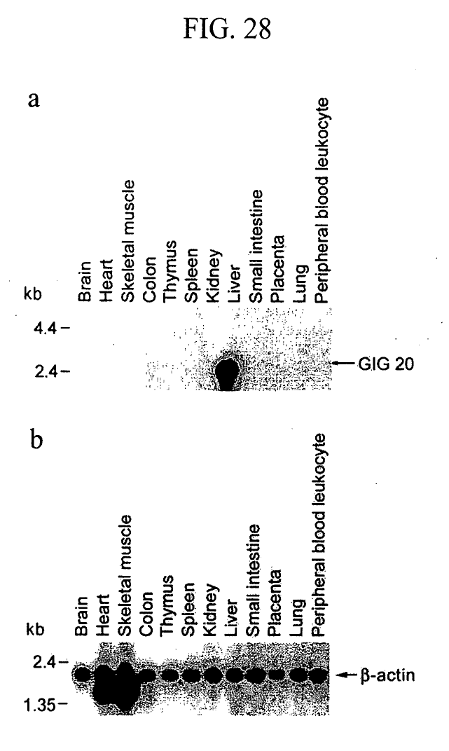

FIG. 28(a) shows a northern blotting result that the GIG20 gene is differentially expressed in various normal tissues, and FIG. 28(b) shows a northern blotting result obtained by hybridizing the same blot with β-actin probe. As shown in FIG. 28(a), dominant GIG20 mRNA transcript having a size of approximately 2.4 kb was overexpressed only in the normal liver tissue.

FIG. 36(a) shows a northern blotting result that the GIG20 gene is differentially expressed in various cancer cell lines, and FIG. 36(b) shows a northern blotting result obtained by hybridizing the same blot with β-actin probe. As shown in FIG. 36(a), the GIG20 gene was not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG20 gene of the present invention had the tumor suppresser function in the normal liver tissue.

3-5. Northern Blotting of GIG22 Gene

In order to assess an expression level of the GIG22 gene, the northern blotting was carried out, as follows.

20 μg of each of the total RNA samples obtained from the three normal liver tissues, the three primary liver cancer tissues and the liver cancer cell line HepG2 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG22 cDNA using the Rediprime II random prime labelling system (Amersham, United Kingdom). The northern blotting procedure was repeated twice; one was quantified using the densitometer and the other was hybridized with the β-actin probe to determine the total mRNA.

FIG. 21(a) shows the northern blotting result that the GIG22 gene is differentially expressed in a normal liver tissue, a primary liver cancer tissue and a liver cancer cell line, and FIG. 21(b) shows the northern blotting result obtained by hybridizing the same blot with β-actin probe. As shown in FIGS. 21(a) and (b), it was revealed that the expression level of the GIG22 gene was highly detected all in the three samples of the normal liver tissue, but its expression level was significantly lower in the three samples of the liver cancer tissue than the normal tissue, and not detected in the one sample of the liver cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

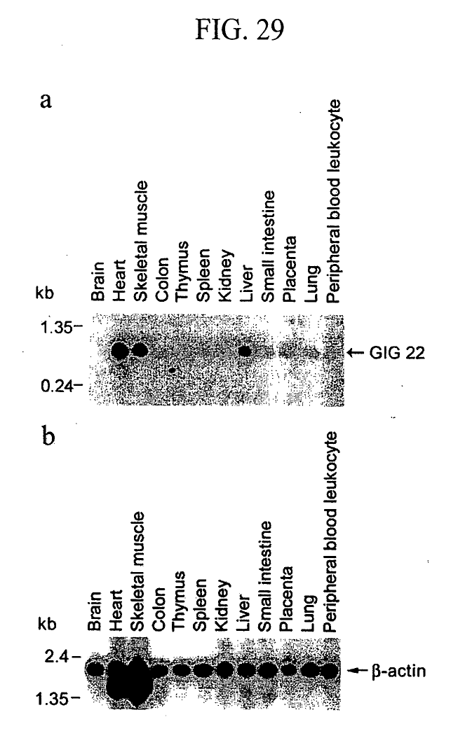

FIG. 29(a) shows a northern blotting result that the GIG22 gene is differentially expressed in various normal tissues, and FIG. 29(b) shows a northern blotting result obtained by hybridizing the same blot with β-actin probe. As shown in FIG. 29(a), dominant GIG22 mRNA transcript having a size of approximately 0.6 kb was overexpressed in the normal tissues such as heart, muscles, liver, kidney, placenta, spleen, lungs, small and large intestines, spleen, thymus and leukocyte.

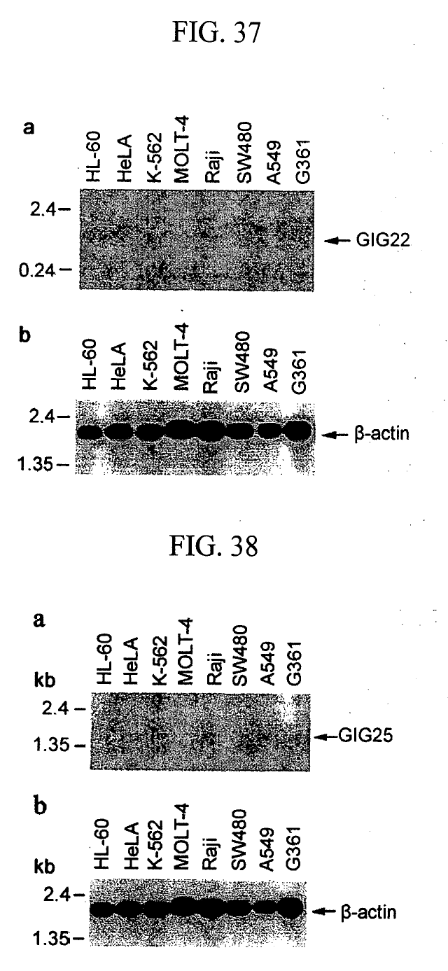

FIG. 37(a) shows a northern blotting result that the GIG22 gene is differentially expressed in various cancer cell lines, and FIG. 37(b) shows a northern blotting result obtained by hybridizing the same blot with β-actin probe. As shown in FIG. 37(a), the GIG22 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG22 gene of the present invention had the tumor suppresser function in the normal tissues such as heart, muscles, liver, kidney, placenta, spleen, lungs, small and large intestines, spleen, thymus and leukocyte. Also, it was revealed that the GIG22 gene of the present invention had the tumor suppresser function, considering that its expression was suppressed in the tissues such as leukemia, uterine cancer, malignant lymphoma, colon cancer, lung cancer, skin cancer, etc. to induce carcinogenesis.

3-6. Northern Blotting of GIG25 Gene

In order to assess an expression level of the GIG25 gene, the northern blotting was carried out, as follows.

20 μg of each of the total RNA samples obtained from the three normal liver tissues, the three primary liver cancer tissues and the liver cancer cell line HepG2 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the HP74 cDNA using the Rediprime II random prime labelling system (Amersham, United Kingdom). The northern blotting procedure was repeated twice; one was quantified using the densitometer and the other was hybridized with the β-actin probe to determine the total mRNA.

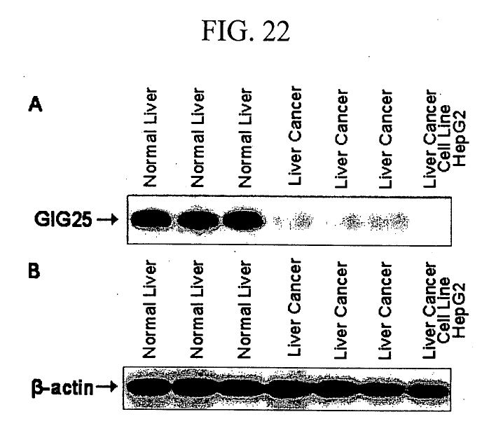

FIG. 22(a) shows the northern blotting result that the GIG25 gene is differentially expressed in a normal liver tissue, a primary liver cancer tissue and a liver cancer cell line, and FIG. 22(b) shows the northern blotting result obtained by hybridizing the same blot with β-actin probe. As shown in FIGS. 22(a) and (b), it was revealed that the expression level of the GIG25 gene was highly detected all in the three samples of the normal liver tissue, but its expression level was not detected in the three samples of the liver cancer tissue and the one sample of the liver cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

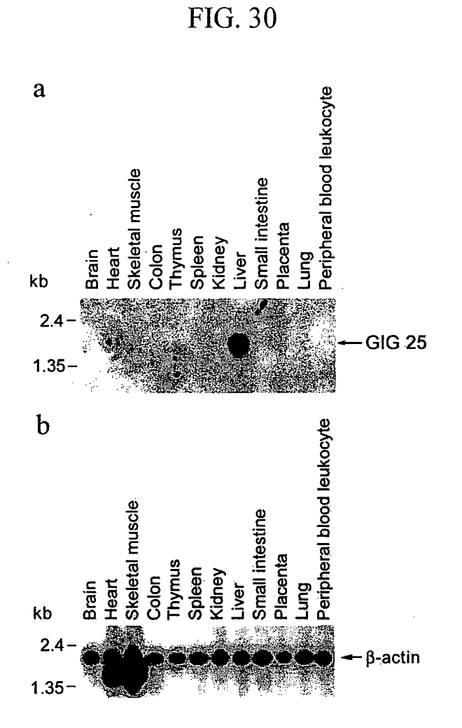

FIG. 30(a) shows a northern blotting result that the GIG25 gene is differentially expressed in various normal tissues, and FIG. 30(b) shows a northern blotting result obtained by hybridizing the same blot with β-actin probe. As shown in FIG. 30(a), dominant GIG25 mRNA transcript having a size of approximately 1.5 kb was overexpressed only in the normal liver tissue.

FIG. 38(a) shows a northern blotting result that the GIG25 gene is differentially expressed in various cancer cell lines, and FIG. 38(b) shows a northern blotting result obtained by hybridizing the same blot with β-actin probe. As shown in FIG. 38(a), the GIG25 gene was not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG25 gene of the present invention had the tumor suppresser function in the normal liver tissue.

3-7. Northern Blotting of GIG36 Gene

In order to assess an expression level of the GIG36 gene, the northern blotting was carried out, as follows.

20 μg of each of the total RNA samples obtained from the three normal breast tissues, the three primary breast cancer tissues and the breast cancer cell line MCF-7 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG36 cDNA using the Rediprime II random prime labelling system (Amersham, United Kingdom). The northern blotting procedure was repeated twice; one was quantified using the densitometer and the other was hybridized with the β-actin probe to determine the total mRNA.

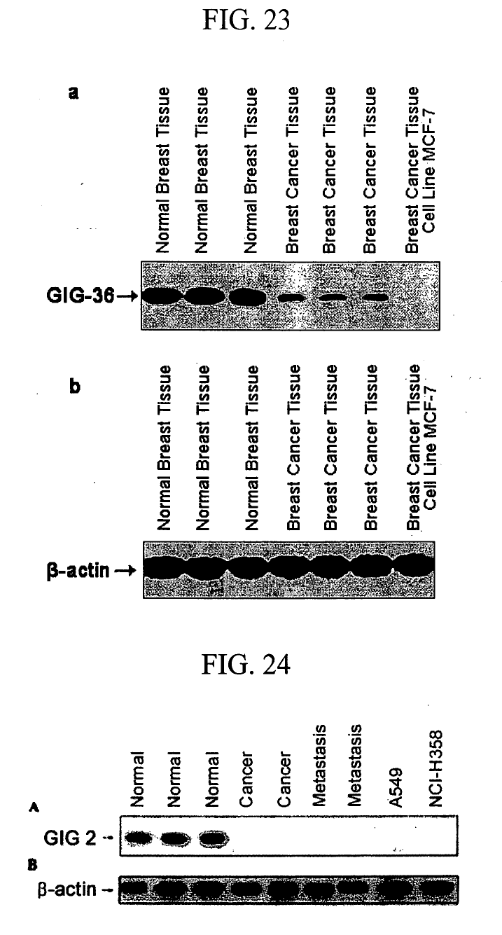

FIG. 23(a) shows the northern blotting result that the GIG36 gene is differentially expressed in a normal breast tissue, a primary breast cancer tissue and a breast cancer cell line, and FIG. 23(b) shows the northern blotting result obtained by hybridizing the same blot with β-actin probe. As shown in FIGS. 23(a) and (b), it was revealed that the expression level of the GIG36 gene was highly detected all in the three samples of the normal breast tissue, but its expression level was significantly lower in the three samples of the breast cancer tissue than the normal tissue, and not detected in the one sample of the breast cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

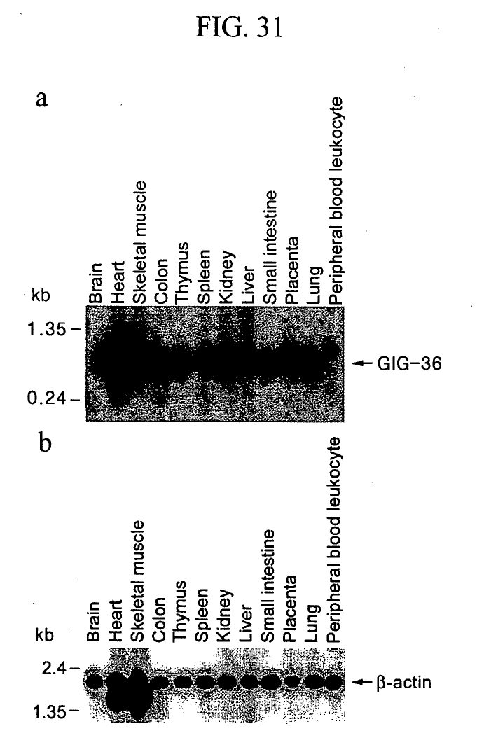

FIG. 31(a) shows a northern blotting result that the GIG36 gene is differentially expressed in various normal tissues, and FIG. 31(b) shows a northern blotting result obtained by hybridizing the same blot with β-actin probe. As shown in FIG. 31(a), dominant GIG36 mRNA transcript having a size of approximately 0.4 kb was overexpressed in the normal tissues such as heart, skeletal muscles, kidney, lungs, small and large intestines, liver, placenta, thymus and spleen.

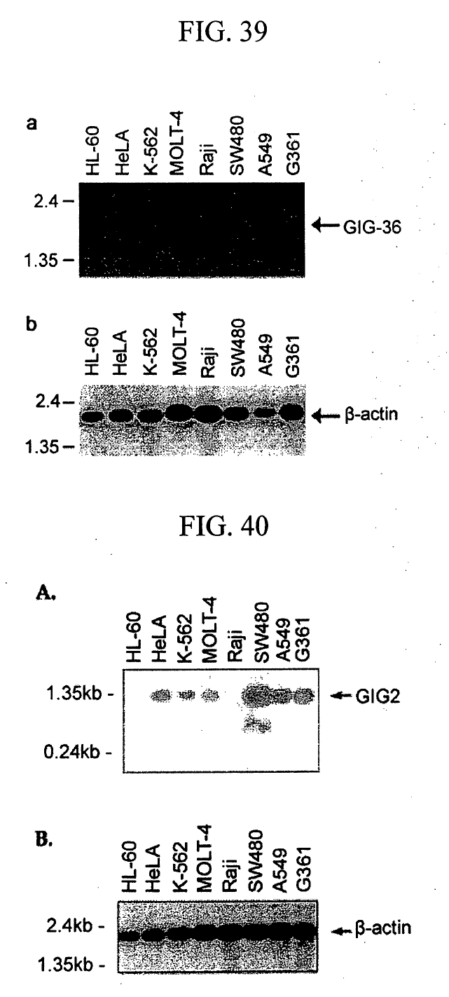

FIG. 39(a) shows a northern blotting result that the GIG36 gene is differentially expressed in various cancer cell lines, and FIG. 39(b) shows a northern blotting result obtained by hybridizing the same blot with β-actin probe. As shown in FIG. 39(a), the GIG36 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. As a result, it was revealed that the GIG36 gene of the present invention had the tumor suppresser function in the normal tissues such as breast, heart, skeletal muscles, kidney, lungs, small and large intestines, liver, placenta, thymus and spleen.

3-8. Northern Blotting of GIG2 Gene

In order to assess an expression level of the GIG2 gene, the northern blotting was carried out, as follows.

20 μg of each of the total RNA samples obtained from the three normal lung tissues, the two primary lung cancer tissues, the two metastatic lung cancer tissues and the lung cancer cell line (A549 and NCI-H358) as described in Example 1-8 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG2 cDNA using the Rediprime II random prime labelling system (Amersham, United Kingdom). The northern blotting procedure was repeated twice; one was quantified using the densitometer and the other was hybridized with the β-actin probe to determine the total mRNA.

FIG. 24(a) shows the northern blotting result that the GIG2 gene is differentially expressed in a normal lung tissue, a primary lung cancer tissue, a metastatic lung cancer tissue and a lung cancer cell line, and FIG. 24(b) shows the northern blotting result obtained by hybridizing the same blot with β-actin probe. As shown in FIGS. 24(a) and (b), it was revealed that the expression level of the GIG2 gene was highly detected all in the three samples of the normal lung tissue, but its expression level was not detected in the two samples of the primary lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

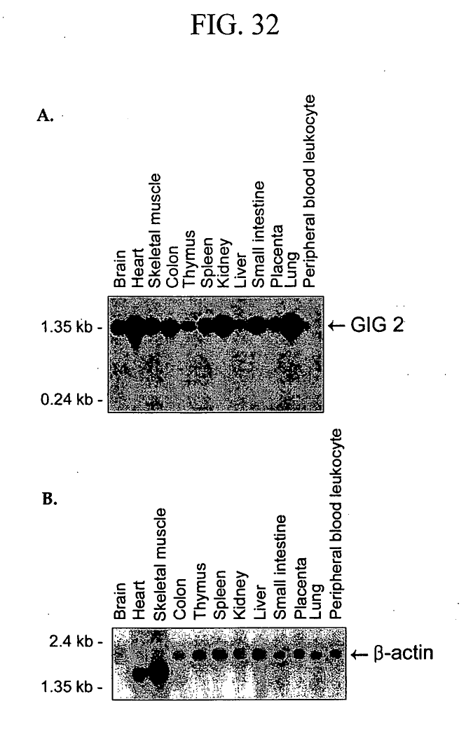

FIG. 32(a) shows a northern blotting result that the GIG2 gene is differentially expressed in various normal tissues, and FIG. 32(b) shows a northern blotting result obtained by hybridizing the same blot with β-actin probe. As shown in FIG. 32(a), dominant GIG2 mRNA transcript having a size of approximately 1.3 kb was very highly overexpressed in the normal tissues such as brain, heart, skeletal muscles, colon, thymus, spleen, kidney, liver, small intestines, placenta, lungs and peripheral blood leukocyte.

FIG. 40(a) shows a northern blotting result that the GIG2 gene is differentially expressed in various cancer cell lines, and FIG. 40(b) shows a northern blotting result obtained by hybridizing the same blot with β-actin probe. As shown in FIG. 40(a), the GIG2 gene was not expressed in the tissues such as promyelocytic leukemia HL-60 and Burkitt's lymphoma (Raji) cell line. As a result, it was revealed that the GIG2 gene of the present invention had the tumor suppresser function in the normal tissues such as brain, heart, skeletal muscles, colon, thymus, spleen, kidney, liver, small intestines, placenta, lungs and peripheral blood leukocyte. Also, it was revealed that the GIG2 gene of the present invention had the tumor suppresser function, considering that its expression was suppressed in the tissues such as uterine cancer, chronic leukemia, colon cancer, lung cancer, skin cancer, etc. to induce carcinogenesis.

### Example 4

### Construction and Transfection of Expression Vector

4-1. GIG12 and GIG36

An expression vector containing a coding region of either GIG12 or GIG36 gene was constructed, as follows. At first, the full-length GIG12 or GIG36 cDNA clone prepared in Example 2 was inserted into a eukaryotic expression vector pcDNA3.1 (Invitrogen, U.S.) to obtain an expression vector pcDNA3.1/GIG12 and an expression vector pcDNA3.1/GIG36, respectively. Each of the expression vectors was transfected into an MCF-7 breast cancer cell line using lipofectamine (Gibco BRL), and then cultured in a DMEM medium including 0.6 mg/ml of G418 (Gibco) to select transfected cells. At this time, MCF-7 cell transfected by the expression vector pcDNA3.1 devoid of the GIG12 cDNA was used as the control group.

4-2. Group of GIG17, GIG19, GIG20, GIG22 and GIG25 Genes

Expression vectors containing each of coding regions of the GIG genes except the GIG12 and the GIG36 genes out of a GIG gene group as described above were constructed, as follows. At first, each of the full-length GIG cDNA clones prepared in Example 2 was inserted into a eukaryotic expression vector pcDNA3.1 (Invitrogen, U.S.) to obtain expression vectors pcDNA3.1/GIG17, pcDNA3.1/GIG19, pcDNA3.1/GIG20, pcDNA3.1/GIG22 and pcDNA3.1/GIG25, respectively. Each of the expression vectors was transfected into an HepG2 liver cancer cell line using lipofectamine (Gibco BRL), and then cultured in a DMEM medium including 0.6 mg/ml of G418 (Gibco) to select transfected cells. At this time, HepG2 cell transfected by the expression vector pcDNA3.1 devoid of the GIG cDNAs was used as the control group.

4-3. GIG2

An expression vector containing a coding region of GIG2 gene was constructed, as follows. At first, the full-length GIG2 cDNA clone prepared in Example 2 was inserted into a eukaryotic expression vector pcDNA3.1 (Invitrogen, U.S.) to obtain an expression vector pcDNA3.1/GIG2. The expression vector was transfected into an A549 lung cancer cell line using lipofectamine (Gibco BRL), and then cultured in a DMEM medium including 0.6 mg/ml of G418 (Gibco) to select transfected cells. At this time, A549 cell transfected by the expression vector pcDNA3.1 devoid of the GIG2 cDNA was used as the control group.

### Example 5

### 5-1. Growth Curve of Breast Cancer Cell Transfected by GIG12 Gene

In order to determine an effect of the GIG12 gene on growth of the breast cancer cell, the wild-type MCF-7 cell, the MCF-7 breast cancer cell transfected by the vector pcDNA3.1/GIG12 prepared in Example 4, and the MCF-7 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

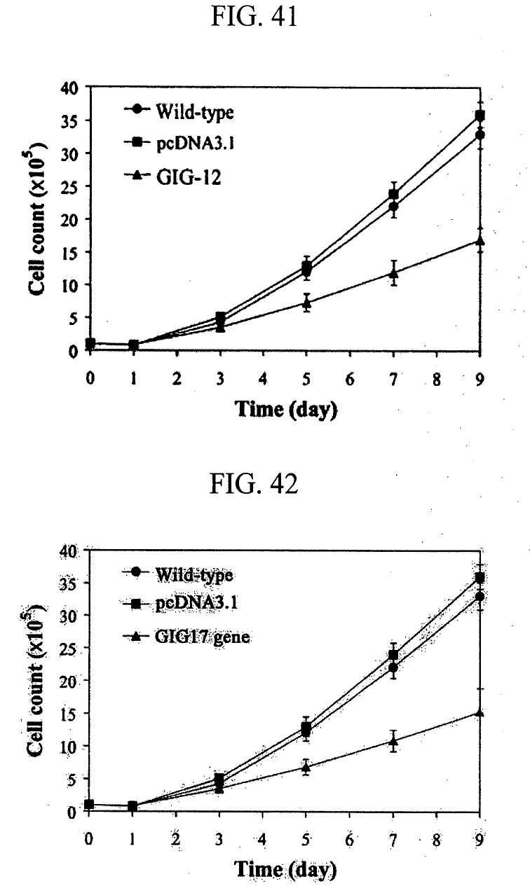

FIG. 41 shows growth curves of the wild-type MCF-7 cell, the MCF-7 breast cancer cell transfected by the vector pcDNA3.1/GIG12 prepared in Example 4, and the MCF-7 cell transfected only by the expression vector pcDNA3.1. As shown in FIG. 41, it was revealed that the MCF-7 breast cancer cell transfected by the vector pcDNA3.1/GIG12 exhibited a higher mortality, compared to those of the MCF-7 cell transfected by the expression vector pcDNA3.1 and the wild-type MCF-7 cell. After 9 days of incubation, only 50% of the MCF-7 breast cancer cell transfected by the vector pcDNA3.1/GIG12 was survived when compared to the wild-type MCF-7 cell. From such a result, it might be seen that the GIG12 gene suppressed growth of the breast cancer cell.

5-2. Growth Curve of Liver Cancer Cell Transfected by GIG17 Gene

In order to determine an effect of the GIG17 gene on growth of the liver cancer cell, the wild-type HepG2 cell, the HepG2 liver cancer cell transfected by the vector cells were counted on days 1, 3, 5, 7 and 9 according to a trypan blue dye exclusion (Freshney, I. R., Culture of Animal Cells, 2nd Ed. A. R. Liss, New York (1987)).

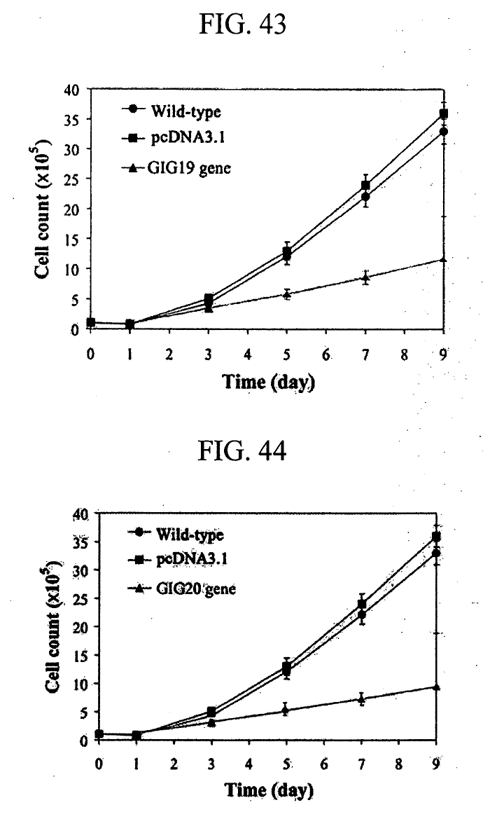

FIG. 43 shows growth curves of the wild-type HepG2 cell, the HepG2 liver cancer cell transfected by the vector pcDNA3.1/GIG19 prepared in Example 4, and the HepG2 cell transfected only by the vector pcDNA3.1. As shown in FIG. 43, it was revealed that the HepG2 liver cancer cell transfected by the vector pcDNA3.1/GIG19 exhibited a higher mortality, compared to those of the HepG2 cell transfected by the expression vector pcDNA3.1 and the wild-type HepG2 cell. After 9 days of incubation, only about 40% of the HepG2 liver cancer cell transfected by the vector pcDNA3.1/GIG19 was survived when compared to the wild-type HepG2 cell. From such a result, it might be seen that the GIG19 gene suppressed growth of the liver cancer cell.

5-4. Growth Curve of Liver Cancer Cell Transfected by GIG20 Gene

In order to determine an effect of the GIG20 gene on growth of the liver cancer cell, the wild-type HepG2 cell, the HepG2 liver cancer cell transfected by the vector pcDNA3.1/GIG20 prepared in Example 4, and the HepG2 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 44 shows growth curves of the wild-type HepG2 cell, the HepG2 liver cancer cell transfected by the vector pcDNA3.1/GIG20 prepared in Example 4, and the pcDNA3.1/GIG17 prepared in Example 4, and the HepG2 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 42 shows growth curves of the wild-type HepG2 cell, the HepG2 liver cancer cell transfected by the vector pcDNA3.1/GIG17 prepared in Example 4, and the HepG2 cell transfected only by the vector pcDNA3.1. As shown in FIG. 42, it was revealed that the HepG2 liver cancer cell transfected by the vector pcDNA3.1/GIG17 exhibited a higher mortality, compared to those of the HepG2 cell transfected by the expression vector pcDNA3.1 and the wild-type HepG2 cell. After 9 days of incubation, only about 45% of the HepG2 liver cancer cell transfected by the vector pcDNA3.1/GIG17 was survived when compared to the wild-type HepG2 cell. From such a result, it might be seen that the GIG17 gene suppressed growth of the liver cancer cell.

5-3. Growth Curve of Liver Cancer Cell Transfected by GIG19 Gene

In order to determine an effect of the GIG19 gene on growth of the liver cancer cell, the wild-type HepG2 cell, the HepG2 liver cancer cell transfected by the vector pcDNA3.1/GIG19 prepared in Example 4, and the HepG2 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived HepG2 cell transfected only by the vector pcDNA3.1. As shown in FIG. 44, it was revealed that the HepG2 liver cancer cell transfected by the vector pcDNA3.1/GIG20 exhibited a higher mortality, compared to those of the HepG2 cell transfected by the expression vector pcDNA3.1 and the wild-type HepG2 cell. After 9 days of incubation, only about 35% of the HepG2 liver cancer cell transfected by the vector pcDNA3.1/GIG20 was survived when compared to the wild-type HepG2 cell. From such a result, it might be seen that the GIG20 gene suppressed growth of the liver cancer cell.

5-5. Growth Curve of Liver Cancer Cell Transfected by GIG22 Gene

In order to determine an effect of the GIG22 gene on growth of the liver cancer cell, the wild-type HepG2 cell, the HepG2 liver cancer cell transfected by the vector pcDNA3.1/GIG22 prepared in Example 4, and the HepG2 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

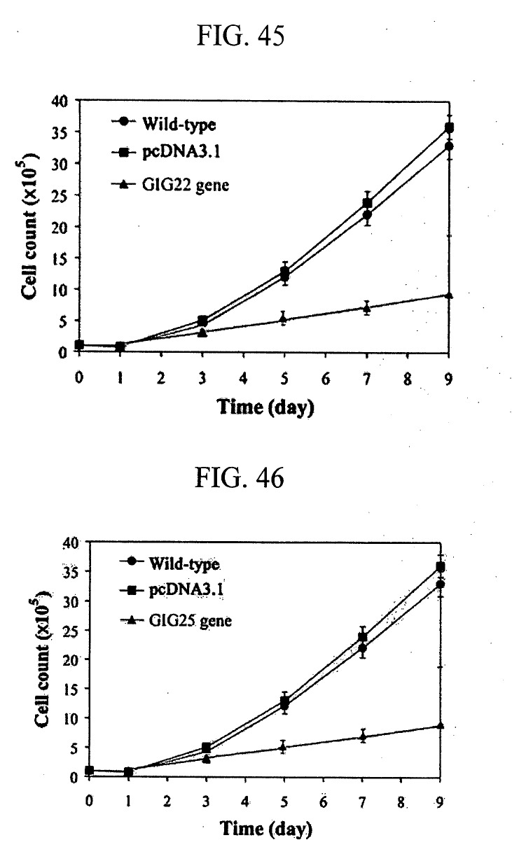

FIG. 45 shows growth curves of the wild-type HepG2 cell, the HepG2 liver cancer cell transfected by the vector pcDNA3.1/GIG22 prepared in Example 4, and the HepG2 cell transfected only by the vector pcDNA3.1. As shown in FIG. 45, it was revealed that the HepG2 liver cancer cell transfected by the vector pcDNA3.1/GIG22 exhibited a higher mortality, compared to those of the HepG2 cell transfected by the expression vector pcDNA3.1 and the wild-type HepG2 cell. After 9 days of incubation, only about 40% of the HepG2 liver cancer cell transfected by the vector pcDNA3.1/GIG22 was survived when compared to the wild-type HepG2 cell. From such a result, it might be seen that the GIG22 gene suppressed growth of the liver cancer cell.

5-6. Growth Curve of Liver Cancer Cell Transfected by GIG25 Gene

In order to determine an effect of the GIG25 gene on growth of the liver cancer cell, the wild-type HepG2 cell, the HepG2 liver cancer cell transfected by the vector pcDNA3.1/GIG25 prepared in Example 4, and the HepG2 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 46 shows growth curves of the wild-type HepG2 cell, the HepG2 liver cancer cell transfected by the vector pcDNA3.1/GIG25 prepared in Example 4, it was HepG2 cell transfected only by the vector pcDNA3.1. As shown in FIG. 46, it was revealed that the HepG2 liver cancer cell transfected by the vector pcDNA3.1/GIG25 exhibited a higher mortality, compared to those of the HepG2 cell transfected by the expression vector pcDNA3.1 and the wild-type HepG2 cell. After 9 days of incubation, only about 35% of the HepG2 liver cancer cell transfected by the vector pcDNA3.1/GIG25 was survived when compared to the wild-type HepG2 cell. From such a result, it might be seen that the GIG25 gene suppressed growth of the liver cancer cell.

5-7. Growth Curve of Breast Cancer Cell Transfected by GIG36 Gene

In order to determine an effect of the GIG36 gene on growth of the breast cancer cell, the wild-type MCF-7 cell, the MCF-7 breast cancer cell transfected by the vector pcDNA3.1/GIG36 prepared in Example 4, and the MCF-7 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

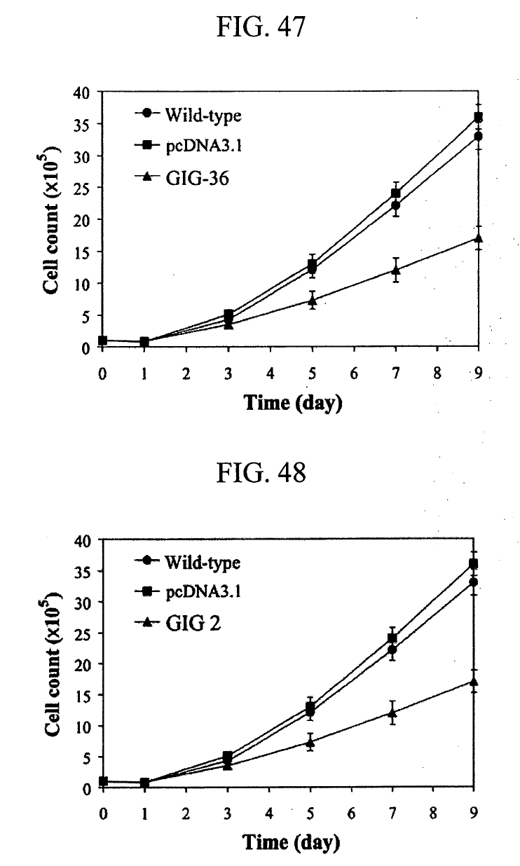

FIG. 47 shows growth curves of the wild-type MCF-7 cell, the MCF-7 breast cancer cell transfected by the vector pcDNA3-1/GIG36 prepared in Example 4, and the MCF-7 cell transfected only by the expression vector pcDNA3.1. As shown in FIG. 47, it was revealed that the MCF-7 breast cancer cell transfected by the vector pcDNA3.1/GIG36 exhibited a higher mortality, compared to those of the MCF-7 cell transfected by the expression vector pcDNA3.1 and the wild-type MCF-7 cell. After 9 days of incubation, only 50% of the MCF-7 breast cancer cell transfected by the vector pcDNA3.1/GIG36 was survived when compared to the wild-type MCF-7 cell. From such a result, it might be seen that the GIG36 gene suppressed growth of the breast cancer cell.

5-8: Growth Curve of Lung Cancer Cell Transfected by GIG2 Gene

In order to determine an effect of the GIG2 gene on growth of the lung cancer cell, the wild-type A549 cell, the A549 lung cancer cell transfected by the vector pcDNA3.1/GIG2 prepared in Example 4, and the A549 cell transfected only by the vector pcDNA3.1 were cultured at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 48 shows growth curves of the wild-type A549 cell, the A549 lung cancer cell transfected by the vector pcDNA3.1/GIG2 prepared in Example 4-3, and the A549 cell transfected only by the expression vector pcDNA3.1. As shown in FIG. 48, it was revealed that the A549 lung cancer cell transfected by the vector pcDNA3.1/GIG2 exhibited a higher mortality, compared to those of the A549 cell transfected by the vector pcDNA3.1 and the wild-type A549 cell. After 9 days of incubation, only about 40% of the A549 lung cancer cell transfected by the vector pcDNA3.1/GIG2 was survived when compared to the wild-type A549 cell. From such a result, it might be seen that the GIG2 gene suppressed growth of the lung cancer cell.

### INDUSTRIAL APPLICABILITY

As described above, the GIG genes of the present invention can be effectively used for diagnosing, preventing and treating the human cancers.

What is claimed is:

1. A human cancer suppressor protein having an amino acid sequence selected from the group consisting of SEQ ID NO: 2, SEQ ID NO: 6, SEQ ID NO: 10, SEQ ID NO: 14, SEQ ID NO: 18, SEQ ID NO: 22, SEQ ID NO: 26 and SEQ ID NO: 30.

2. The human cancer suppressor protein according to claim 1, wherein the cancer is a cancer of the normal tissue selected from the group consisting of breast, lungs, thymus, liver, skeletal muscles, kidney, spleen, heart, placenta and peripheral blood.

3. A human cancer suppressor gene having a DNA sequence selected from the group consisting of SEQ ID NO: 1, SEQ ID NO: 5, SEQ ID NO, 9, SEQ ID NO: 13, SEQ ID NO: 17, SEQ ID NO: 21, SEQ ID NO: 25 and SEQ ID NO: 29, which encode the corresponding proteins.

4. The human cancer suppressor genes according to claim 3, wherein the cancer is a cancer of the normal tissue selected from the group consisting of breast, lungs, thymus, liver, skeletal muscles, kidney, spleen, heart, placenta and peripheral blood.

5. An expression vector containing each of the genes as defined in claim 3.

6. A cell transformed by each of the expression vectors as defined in claim 5.

7. The cell according to claim 6, wherein it is microorganisms or animal cells.

8. The cell according to claim 7, wherein the cell is selected from the group consisting of Escherichia coli DH5α/GIG12/pcDNA3.1 (Accession No. KCTC 10642BP), E. coli DH5α/GIG17/pcDNA3.1 (Accession No. KCTC 10655BP), E. coli DH5α/GIG19/pcDNA3.1 (Accession No. KCTC 10656BP), E. coli DH5α/GIG20/pcDNA3.1 (Accession No. KCTC 10657BP), E. coli DH5α/GIG22/pcDNA3.1 (Accession No. KCTC 10658BP), E. coli DH5α/GIG25/pcDNA3.1 (Accession No. KCTC 10659BP), E. coli DH5α/GIG36/pcDNA3.1 (Accession No: KCTC 10643BP) and E. coli DH5α/GIG2/pcDNA3.1 (Accession No. KCTC 10641 BP).


SEQUENCE LISTING (32 sequences)

SEQ ID NO: 1 - DNA, 2291 bp, Homo sapiens
agggaaggggtgtctattgggcaacagggcggcaaagccctgaataaaggggcgcagggcaggcgcaagtgcagagccttcgtttgccaagtcgcctccagaccgcagacatgaaacttgtcttcctcgtcctgctgttcctcggggccctcggactgtgtctggctggccgtaggagaaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggtcttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgacgcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattc

SEQ ID NO: 2 - protein, 711 aa, Homo sapiens [sequence omitted]

SEQ ID NO: 3 - DNA, 13 bp, Artificial Sequence
  Primer H-AP32
aagcttcctgcaa

SEQ ID NO: 4 - DNA, 16 bp, Artificial Sequence
  anchored oligo-dT primer
aagctttttttttttc

SEQ ID NO: 5 - DNA, 1145 bp, Homo sapiens
gtgcctactgccctctcttgccgcccgcacctgcagccccgcacctgccgcttgcacctgctctacccggttcaagcatggctgaccaggcgcccttcgacacggacgtcaacaccctgacccgcttcgtcatggaggagggcaggaaggcccgcggcacgggcgagttgacccagctgctcaactcgctctgcacagcagtcaaagccatctcttcggcggtgcgcaaggcgggcatcgcgcacctctatggcattgctggttctaccaacgtgacaggtgatcaagttaagaagctggacgtcctctccaacgacctggttatgaacatgttaaagtcatcctttgccacgtgtgttctcgtgtcagaagaagataaacacgccatcatagtggaaccggagaaaaggggtaaatatgtggtctgttttgatccccttgatggatcttccaacatcgattgccttgtgcagccccgcgtccgttggaaccatttttggcatctatagaaagaaatcaactgatgagccttctgagaaggatgctctgcaaccaggccggaacctggtggcagccggctacgcactgtatggcagtgccaccatgctggtccttgccatggactgtggggtcaactgcttcatgctggacccggccatcggggagttcattttggtggacaaggatgtgaagataaaaaagaaaggtaaaatctacagccttaacgagggctacgccaaggactttgaccctgccgtcactgagtacatccagaggaagaagttccccccagataattcagctccttatggggcccggtatgtgggctccatggtggctgatgttcatcgcactctggtctacggagggatatttctgtaccccgctaacaagaagagccccaatggaaagctgagactgctgtacgaatgcaaccccatggcctacgtcatggagaaggctgggggaatggccaccactgggaaggaggccgtgttagacgtcattcccacagacattcaccagagggcgccggtgatcttggggtcccccgacgacgtgctcgagttcctgaaggtgtatgagaagcactctgcccagtgagcacctgccctgcctgcatctggagaattgcctctacctgg

SEQ ID NO: 6 - protein, 338 aa, Homo sapiens
MADQAPFDTDVNTLTRFVMEEGRKARGTGELTQLLNSLCTAVKAISSAVRKAGIAHLYGIAGSTNVTGDQVKKLDVLSNDLVMNMLKSSFATCVLVSEEDKHAIIVEPEKRGKYVVCFDPLDGSSNIDCLVSVGTIFGIYRKKSTDEPSEKDALQPGRNLVAAGYALYGSATMLVLAMDCGVNCFMLDPAIGEFILVDKDVKIKKKGKIYSLNEGYAKDFDPAVTEYIQRKKFPPDNSAPYGARYVGSMVADVHRTLVYGGIFLYPANKKSPNGKLRLLYECNPMAYVMEKAGGMATTGKEAVLDVIPTDIHQRAPVILGSPDDVLEFLKVYEKHSAQ

SEQ ID NO: 7 - DNA, 13 bp, Artificial Sequence
  Primer H-AP7
aagcttaacgagg

SEQ ID NO: 8 - DNA, 16 bp, Artificial Sequence
  anchored oligo-dT primer
aagctttttttttttg

SEQ ID NO: 9 - DNA, 1155 bp, Homo sapiens
tggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtg

SEQ ID NO: 10 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 11 - DNA, 13 bp, Artificial Sequence
  Primer H-AP40
aagcttgtcagcc

SEQ ID NO: 12 - DNA, 16 bp, Artificial Sequence
  anchored oligo-dT primer
aagctttttttttttc

SEQ ID NO: 13 - DNA, 1363 bp, Homo sapiens
tggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcggatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagcca

SEQ ID NO: 14 - protein, 417 aa, Homo sapiens
MKWVTFISLLFLFSSAYSRGVFRRDAHKSEVAHRFKDLGEENFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 15 - DNA, 13 bp, Artificial Sequence
  Primer H-AP40
aagcttgtcagcc

SEQ ID NO: 16 - DNA, 16 bp, Artificial Sequence
  anchored oligo-dT primer
aagctttttttttttc

SEQ ID NO: 17 - DNA, 550 bp, Homo sapiens
tggatctgcgagaagaaaccgcgctaactagtttgtccctacggccgcctcgtagtcactgccgcggcgccttgagtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggcctgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgc

SEQ ID NO: 18 - protein, 150 aa, Homo sapiens
MAARGVIAPVGESLRYAEYLQPSAKRPDADVDQQRLVRSLIAVGLGVAALAFAGRYAFRIWKPLEQVITETAKKISTPSFSSYYKGGFEQKMSRREAGLILGVSPSAGKAKIRTAHRRVMILNHPDKGGSPYVAAKINEAKDLLETTTKH

SEQ ID NO: 19 - DNA, 13 bp, Artificial Sequence
  Primer H-AP30
aagcttcgtacgt

SEQ ID NO: 20 - DNA, 16 bp, Artificial Sequence
  anchored oligo-dT primer
aagctttttttttttc

SEQ ID NO: 21 - DNA, 1474 bp, Homo sapiens
gctacatccagctccctgagagttgagaatggagagaatgtacctctcctgactctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaagaaccttgactcgcagacaatgatggtcctggtgaattacatcttctttaaagccaaatgggagatgccctttgacccccaagatactcatcagtcaaggttctacttgaacaagaaaaagtgggtaatggtacccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgtagctctcacatgcacaggg

SEQ ID NO: 22 - protein, 287 aa, Homo sapiens [sequence omitted]

SEQ ID NO: 23 - DNA, 13 bp, Artificial Sequence
  Primer H-AP40
aagcttgtcagcc

SEQ ID NO: 24 - DNA, 16 bp, Artificial Sequence
  anchored oligo-dT primer
aagctttttttttttc

SEQ ID NO: 25 - DNA, 394 bp, Homo sapiens
aggacgaaaccatgaagagcctgatccttcttgccatcctggccgccttagcggtagtaactttgtgttatgaatcacatgaaagcatggaatcttatgaacttaatcccttcattaacaggagaaatgcaaataccttcatatcccctcagcagagatggagagctaaagtccaagagaggatccgagaacgctctaagcctgtccacgagctcaatagggaagcctgtgatgactacagactttgcgaacgctacgccatggtttatggatacaatgctgcctataatcgctacttcaggaagcgccgagggaccaaatgagactgagggaagaaaaaaaatctctttttttctggaggctggcacctgattttgtatccccctgtagcagc

SEQ ID NO: 26 - protein, 103 aa, Homo sapiens
MKSLILLAILAALAVVTLCYESHESMESYELNPFINRRNANTFISPQQRWRAKVQERIRERSKPVHELNREACDDYRLCERYAMVYGYNAAYNRYFRKRRGTK

SEQ ID NO: 27 - DNA, 13 bp, Artificial Sequence
  Primer H-AP29
aagcttagcagca

SEQ ID NO: 28 - DNA, 16 bp, Artificial Sequence
  anchored oligo-dT primer
aagctttttttttttc

SEQ ID NO: 29 - DNA, 705 bp, Homo sapiens
ctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctaga

SEQ ID NO: 30 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 31 - DNA, 13 bp, Artificial Sequence
  Primer H-AP32
aagcttcttgcaa

SEQ ID NO: 32 - DNA, 16 bp, Artificial Sequence
  anchored oligo-dT primer
aagctttttttttttg